(19) United States
(12) Patent Application Publication
(10) Pub. No.: US 2026/0184670 A1
(43) Pub. Date: Jul. 2, 2026

(54) ORGANIC COMPOUNDS AND ORGANIC LIGHT EMITTING DIODE COMPRISING THE SAME

(71) Applicant: Material Science Co., Ltd., Hwaseong-si (KR)

(72) Inventors: Hee Jun PARK, Hwaseong-si (KR); Gi Rim LEE, Hwaseong-si (KR); Seung Hyun KIM, Hwaseong-si (KR); Ki Hoon SHIN, Hwaseong-si (KR)

(21) Appl. No.: 19/435,096

(22) Filed: Dec. 29, 2025

(30) Foreign Application Priority Data
Dec. 30, 2024 (KR) 10-2024-0199547
Dec. 22, 2025 (KR) 10-2025-0207009

(51) Int. Cl. C07C 233/58; C07C 69/75; C09D 7/63; H10K 59/80

(52) U.S. Cl. CPC C07C 233/58; C07C 69/75; C09D 7/63; H10K 59/879

(57) ABSTRACT

The present disclosure relates to a novel organic compound represented by Chemical Formula 1 and an organic light emitting diode including the same. An organic light emitting diode according to an embodiment of the present disclosure includes a first electrode, a second electrode facing the first electrode, one or more organic layers disposed between the first electrode and second electrode, and a capping layer disposed on an outer side of one or more of the first electrode and second electrode, wherein the capping layer includes the novel organic compound represented by Chemical Formula 1.

### CROSS-REFERENCE TO RELATED APPLICATION

This application claims priority from Korean Patent Application No. 10-2024-0199547 filed on Dec. 30, 2024 and Korean Patent Application No. 10-2025-0207009 filed on Nov. 22, 2025 in the Korean Intellectual Property Office, and all the benefits accruing therefrom under 35 U.S.C. 119, the contents of which in its entirety are herein incorporated by reference.

### TECHNICAL FIELD

The present disclosure relates to an organic compound and an organic light emitting diode comprising the same.

### BACKGROUND

Organic light emitting diodes (OLEDs) have been actively developed and commercialized as a light source for flat panel displays such as wall-mountable televisions, backlights for displays, lighting devices, and signboards, because they have simplified structure, various advantages in manufacturing processes, high luminance, excellent viewing angle characteristics, a fast response speed, and a low driving voltage, compared to other flat panel display devices such as conventional liquid crystal displays (LCDs), plasma display panels (PDPs), and field emission displays (FEDs).

The OLED includes two electrodes and an organic layer disposed between the two electrodes. The OLED is a device that operates on the principle that electrons and holes are injected into a light emitting layer from the two electrodes, respectively, and recombine within the light emitting layer to generate excitons, and light is generated when the generated excitons transition from an excited state to a ground state.

The OLED may include at least one light emitting layer. In general, an OLED having a plurality of light emitting layers may include light emitting layers that emit light having different peak wavelengths, thereby enabling a specific color to be realized through a combination of light having different peak wavelengths.

These OLEDs may be categorized into a bottom-emitting structure and a top-emitting structure. The bottom-emitting OLED uses a reflective cathode to emit light generated in the light emitting layer toward a translucent anode. In contrast, the top-emitting OLED uses a reflective anode to direct the light generated in the light emitting layer—after being reflected at the anode—toward the transparent cathode, which faces the driving thin-film transistor.

As display devices have advanced, the need for a capping layer compound that can improve the luminous efficiency and lifetime of OLEDs, has increased. Conventionally, high-refractive-index compounds have been used to diffuse light emitted from the panel, thereby enhancing light transmittance and suppressing light absorption within the device to improve device efficiency. However, to enhance light efficiency, the necessity for low-refractive index compounds that can reconverge the light diffused by high-refractive index compounds and transmit it to the display, thereby improving the device efficiency, has increased.

### RELATED ART DOCUMENT

### Patent Document

(Patent Document 1) JP2002-083685A
(Patent Document 2) CN101096357B
(Patent Document 3) KR2023-0090431A

### SUMMARY

An object of the present disclosure is to provide a novel organic compound and an organic light emitting diode comprising the same.

Embodiments according to the present disclosure may be used to achieve other problems not specifically mentioned, in addition to the above problems.

The present disclosure is not limited to the objects described above, and other objects and advantages of the present disclosure not mentioned, can be understood from the following description and will be more clearly understood from the embodiments of the present disclosure. Furthermore, it will be readily apparent that the objects and advantages of the present disclosure may be realized by means and combinations thereof set forth in the claims.

An embodiment of the present disclosure may provide a compound represented by the following Chemical Formula 1, and the definition of Chemical Formula 1 is identical to that described in the present specification and claims.

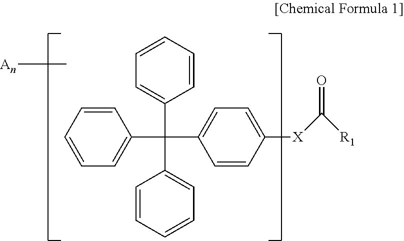

wherein in the chemical formula 1,
n is an integer from 1 to 19,
X is O or N(R₂),
A, R₁, and R₂ are identical to or different from each other, and each of A, R₁, and R₂ is independently selected from the group consisting of hydrogen, a substituted or unsubstituted alkyl group having 1 to 30 carbon atoms, and a substituted or unsubstituted cycloalkyl group having 3 to 20 carbon atoms,
when one or more of A, R₁, and R₂ are substituted, each of A, R₁, and R₂ is independently substituted with one or more substituents selected from the group consisting of deuterium, a cyano group, a halogen group, an alkyl group having 1 to 30 carbon atoms, a cycloalkyl group having 3 to 20 carbon atoms, an aryl group having 6 to 30 carbon atoms, an arylalkyl group having 7 to 30 carbon atoms, a heteroaryl group having 5 to 60 carbon atoms, a heteroarylalkyl group having 6 to 60 carbon atoms, an amine group, an alkylamino group having 1 to 30 carbon atoms, an arylalkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms, and when a plurality of substituents are present, each substituent may be identical to or different from each other.

An organic light emitting diode according to another embodiment of the present disclosure may include: a first electrode, a second electrode facing the first electrode, one or more organic layers disposed between the first electrode and second electrode, and a capping layer disposed on an outer side of one or more of the first electrode and second electrode, wherein the capping layer may include the compound represented by Chemical Formula 1.

The compound represented by Chemical Formula 1 of the present disclosure has a low refractive index of about 1.50 or more and 1.70 or less and a high transmittance of about 80% or more, at wavelengths of 400 nm to 650 nm.

In addition, an organic light emitting diode including the compound represented by Chemical Formula 1 of the present disclosure exhibits excellent characteristics, such as driving voltage, luminous efficiency, external quantum efficiency (EQE), lifetime, and stability.

The effects of the present specification are not limited to those described above, and other effects not mentioned will be clearly understood by those skilled in the art from the entirety of the present specification.

The above effects and additional effects will be described in detail below.

### DETAILED DESCRIPTION

The aforementioned objects, features, and advantages will be described in detail below, and accordingly, those skilled in the art to which the present disclosure pertains will be able to easily implement the technical spirit of the present disclosure.

In describing this specification, the detailed descriptions of related known technologies will be omitted if they are deemed to unnecessarily obscure the gist of the present disclosure.

As used herein, it is to be understood that when the terms such as “comprises,” “has,” “consists of,” “arranges,” “provides,” etc. are used with respect to components, additional components may be present, unless the term “only” is used. Also, it is to be understood that, unless expressly stated otherwise, when a component is referred to in the singular, it is intended to include the plural.

In interpreting the components in the present specification, it is to be understood that the ranges include allowable tolerances even if not explicitly stated.

As used herein, when any configuration is described as being disposed “on (or under)” a component or “on an upper portion (or lower portion)” of a component, it may mean not only that any configuration is disposed directly contacting the top (or bottom) surface of the component, but also that another configuration may be interposed between the component and any configuration disposed on (or under) the component.

As used herein, the term “halogen group” includes fluorine, chlorine, bromine, and iodine.

As used herein, the term “alkyl group” refers to both a straight-chain alkyl radical and a branched-chain alkyl radical. Unless otherwise specified, the alkyl group contains 1 to 30 carbon atoms and may include, but is not limited to, methyl, ethyl, propyl, isopropyl, butyl, sec-butyl, isobutyl, tert-butyl, pentyl, isoamyl, hexyl, etc. Additionally, the alkyl group may be optionally substituted.

As used herein, the term “cycloalkyl group” refers to a cyclic alkyl radical. Unless otherwise specified, the cycloalkyl group contains 3 to 20 carbon atoms and may include, but is not limited to, cyclopropyl, cyclobutyl, cyclopentyl, cyclohexyl, norbornyl, adamantyl, etc. Additionally, the cycloalkyl group may be optionally substituted.

As used herein, the terms “aralkyl group” and “arylalkyl group” are used interchangeably, and refers to an alkyl group having an aromatic group as a substituent. Additionally, the aralkyl (arylalkyl) group may be optionally substituted.

As used herein, the terms “aryl group” and “aromatic group” are used interchangeably, and the aryl group includes both monocyclic and polycyclic groups. A polycyclic ring may include a “fused ring,” which is two or more rings having two carbon atoms common to two adjacent rings. It may also include a form in which two or more rings are simply attached to each other or fused together. Unless otherwise specified, the aryl group contains 6 to 30 carbon atoms, and may include, but is not limited to, phenyl, biphenyl, terphenyl, naphthyl, phenanthryl, anthryl, fluorenyl, dimethylfluorenyl, diphenylfluorenyl, spirofluorenyl, etc. Additionally, the aryl group may be optionally substituted.

As used herein, the terms “heteroaryl group” and “heteroaromatic group” are used interchangeably, and the heteroaryl group includes both monocyclic and polycyclic groups. A polycyclic ring may include a “fused ring,” which is two or more rings having two carbon atoms or heteroatoms common to two adjacent rings. It may also include a form in which two or more rings are simply attached to each other or fused together. Unless otherwise specified, the heteroaryl group may contain 5 to 60 carbon atoms, wherein one or more carbon atoms in the ring are substituted with heteroatoms such as oxygen (O), nitrogen (N), sulfur (S), or selenium (Se). The heteroaryl group may include, but is not limited to, a 6-membered monocyclic ring such as pyridinyl, pyrazinyl, pyrimidinyl, pyridazinyl, and triazinyl, a polycyclic ring such as phenoxathiinyl, indolizinyl, indolyl, purinyl, quinolyl, isoquinolyl, benzoxazolyl, benzothiazolyl, benzimidazolyl, benzofuranyl, dibenzofuranyl, benzothiophenyl, dibenzothiophenyl, phenylcarbazolyl, 9-phenylcarbazolyl, and carbazolyl, as well as 2-furanyl, N-imidazolyl, 2-isoxazolyl, 2-pyridyl, 2-pyrimidyl, etc. Additionally, the heteroaryl group may be optionally substituted.

As used herein, the term “carbon ring” may be used to encompass both a “cycloalkyl group,” which is an alicyclic ring group, and an “aryl group (aromatic group),” which is an aromatic ring group, unless otherwise specified.

As used herein, the terms “heteroalkyl group” and “heteroarylalkyl group” refer to an alkyl group or an arylalkyl group in which one or more of carbon atoms constituting the group are substituted with heteroatoms such as oxygen (O), nitrogen (N), sulfur (S), or selenium (Se). Additionally, the heteroalkyl group and the heteroarylalkyl group may be optionally substituted.

As used herein, the terms “alkylamino group,” “arylalkylamino group,” “arylamino group,” and “heteroarylamino group” refer to an alkyl group, an arylalkyl group, an aryl group, or a heteroaryl group, which is a heterocyclic ring, substituted with an amino group, and are meant to include primary, secondary, and tertiary amines. Additionally, the alkylamino group, the arylalkylamino group, the arylamino group, and the heteroarylamino group may be optionally substituted.

As used herein, the terms “alkylsilyl group,” “arylsilyl group,” “alkoxy group,” “aryloxy group,” “alkylthio group,” and “arylthio group” refer to an alkyl group and an aryl group substituted with a silyl group, an oxy group, and a thio group, respectively. Additionally, the alkylsilyl group, the arylsilyl group, the alkoxy group, the aryloxy group, the alkylthio group, and arylthio group may be optionally substituted.

As used herein, the terms “arylene group,” “arylalkylene group,” “heteroarylene group,” and “heteroarylalkylene group” refer to divalent substituent derived from the respective aryl group, arylalkyl group, heteroaryl group, and heteroarylalkyl group, each having one additional point of substitution. Additionally, the arylene group, the arylalkylene group, the heteroarylene group, and the heteroarylalkylene group may be optionally substituted.

As used herein, the term “substitution” means that a hydrogen (H) atom bonded to a carbon or nitrogen atom, etc., included in the compound structure of the present disclosure is replaced with a substituent other than hydrogen. When a plurality of substituents are present, the substituents may be identical to or different from each other.

The substituents are each independently substituted with one or more substituents selected from the group consisting of deuterium, a cyano group, a halogen group, an alkyl group having 1 to 30 carbon atoms, a cycloalkyl group having 3 to 20 carbon atoms, an aryl group having 6 to 30 carbon atoms, an arylalkyl group having 7 to 30 carbon atoms, a heteroaryl group having 5 to 60 carbon atoms, a heteroarylalkyl group having 6 to 60 carbon atoms, an amine group, an alkylamino group having 1 to 30 carbon atoms, an arylalkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms, and when a plurality of substituents are present, the substituents are identical to or different from each other, and may bond to an adjacent group to form a substituted or unsubstituted ring.

Each target and substituent defined herein may be identical to or different from each other, unless otherwise specified.

The units used herein are based on weight (wt), unless otherwise specified. For example, if “%” is stated, it is to be interpreted as weight percent (wt %).

Hereinafter, an organic compound according to the present disclosure and an organic light emitting diode comprising the same will be described in detail.

The organic compound according to the present disclosure may be represented by the following Chemical Formula 1:

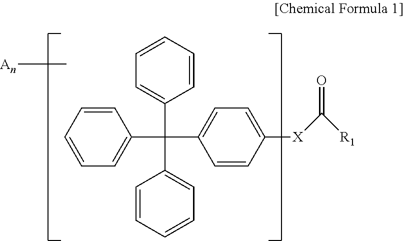

wherein in the chemical formula 1,
n is an integer from 1 to 19,
X is O or N(R₂),
A, R₁, and R₂ are identical to or different from each other, and each of A, R₁, and R₂ is independently selected from the group consisting of hydrogen, a substituted or unsubstituted alkyl group having 1 to 30 carbon atoms, and a substituted or unsubstituted cycloalkyl group having 3 to 20 carbon atoms,
when one or more of A, R₁, and R₂ are substituted, each of A, R₁, and R₂ is independently substituted with one or more substituents selected from the group consisting of deuterium, a cyano group, a halogen group, an alkyl group having 1 to 30 carbon atoms, a cycloalkyl group having 3 to 20 carbon atoms, an aryl group having 6 to 30 carbon atoms, an arylalkyl group having 7 to 30 carbon atoms, a heteroaryl group having 5 to 60 carbon atoms, a heteroarylalkyl group having 6 to 60 carbon atoms, an amine group, an alkylamino group having 1 to 30 carbon atoms, an arylalkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms, and when a plurality of substituents are present, the substituents are identical to or different from each other.

An organic light emitting diode according to an embodiment of the present disclosure includes a first electrode, a second electrode facing the first electrode, one or more organic layers disposed between the first electrode and second electrode, and a capping layer disposed on an outer side of one or more of the first electrode and second electrode. The capping layer includes a compound represented by Chemical Formula 1. A detailed description of each electrode and layer in the organic light emitting diode will be provided below.

According to an example, in Chemical Formula 1, n means the number of A in chemical formula 1, may be, for example, an integer of 1 to 8, 1 to 6, 1 to 4, 1 to 3, 2 to 8, 2 to 6, 2 to 4, or 2 to 3, 3 to 8, 3 to 6, 3 to 4, 3, or 6. For example, when n is smaller, that is, when a compound has fewer substituents on a tetraphenyl moiety, the molecular weight tends to be relatively lower, allowing the process to be carried out at a lower temperature. Furthermore, as the value of n decreases, the compound molecule represented by Chemical Formula 1 exhibits a tendency toward reduced hydrophobic properties, which in turn provides an advantage of increased adhesion to other hydrophilic compounds and/or to an electrode.

According to an example, in Chemical Formula 1, A, R₁, and R₂ are identical to or different from each other, and each of A, R₁, and R₂ may be independently selected from the group consisting of hydrogen, a substituted or unsubstituted alkyl group having 1 to 15 carbon atoms, for example, 1 to 10 carbon atoms, for example, 1 to 6 carbon atoms, or a substituted or unsubstituted cycloalkyl group having 3 to 15 carbon atoms, for example, 3 to 10 carbon atoms, for example, 3 to 6 carbon atoms. Here, when substituted, it may be substituted with one or more substituents selected from the group consisting of an alkyl group having 1 to 30 carbon atoms, a cycloalkyl group having 3 to 20 carbon atoms, an aryl group having 6 to 30 carbon atoms, an arylalkyl group having 7 to 30 carbon atoms, a heteroaryl group having 5 to 60 carbon atoms, and a heteroarylalkyl group having 6 to 60 carbon atoms.

According to an example, in Chemical Formula 1, A, R₁, and R₂ are identical to or different from each other, and each of A, R₁, and R₂ may be independently selected from a substituted or unsubstituted methyl group, a substituted or unsubstituted ethyl group, a substituted or unsubstituted propyl group, a substituted or unsubstituted isopropyl group, a substituted or unsubstituted butyl group, a substituted or unsubstituted sec-butyl group, a substituted or unsubstituted tert-butyl group, a substituted or unsubstituted isobutyl group, a substituted or unsubstituted 2-ethylbutyl group, a substituted or unsubstituted 3,3-dimethylbutyl group, a substituted or unsubstituted pentyl group, a substituted or unsubstituted isopentyl group, a substituted or unsubstituted neopentyl group, a substituted or unsubstituted tert-pentyl group, a substituted or unsubstituted 1-methylpentyl group, a substituted or unsubstituted 3-methylpentyl group, a substituted or unsubstituted 4-methyl-2-pentyl group, a substituted or unsubstituted hexyl group, a substituted or unsubstituted cyclopentyl group, a substituted or unsubstituted tert-butylcyclopentyl group, a substituted or unsubstituted cyclohexyl group, a substituted or unsubstituted tert-butylcyclohexyl group, a substituted or unsubstituted 4-methylcyclohexyl group, a substituted or unsubstituted norbornyl group, a substituted or unsubstituted adamantly group, a substituted or unsubstituted phenyl group, a substituted or unsubstituted biphenyl group, or a substituted or unsubstituted terphenyl group, etc. Here, when any of A, R₁, and R₂ are substituted, they may be substituted with one or more substituents selected from the group consisting of an alkyl group having 1 to 30 carbon atoms, a cycloalkyl group having 3 to 20 carbon atoms, an aryl group having 6 to 30 carbon atoms, an arylalkyl group having 7 to 30 carbon atoms, a heteroaryl group having 5 to 60 carbon atoms, and a heteroarylalkyl group having 6 to 60 carbon atoms.

According to an example, in Chemical Formula 1, R₁ and R₂ are identical to or different from each other, and each of R₁ and R₂ may be independently selected from the group consisting of hydrogen, a substituted or unsubstituted alkyl group having 1 to 15 carbon atoms, a substituted or unsubstituted alkyl group having 1 to 10 carbon atoms, a substituted or unsubstituted alkyl group having 1 to 6 carbon atoms, a substituted or unsubstituted cycloalkyl group having 3 to 15 carbon atoms, a substituted or unsubstituted cycloalkyl group having 3 to 10 carbon atoms, and a substituted or unsubstituted cycloalkyl group having 3 to 6 carbon atoms.

According to an example, in Chemical Formula 1, R₁ may be selected from the group consisting of a substituted or unsubstituted alkyl group having 1 to 15 carbon atoms, a substituted or unsubstituted alkyl group having 1 to 10 carbon atoms, a substituted or unsubstituted alkyl group having 1 to 6 carbon atoms, a substituted or unsubstituted cycloalkyl group having 3 to 15 carbon atoms, a substituted or unsubstituted cycloalkyl group having 3 to 10 carbon atoms and a substituted or unsubstituted cycloalkyl group having 3 to 6 carbon atoms.

According to an example, in Chemical Formula 1, R₂ may be, for example, hydrogen, a substituted or unsubstituted alkyl group having 1 to 15 carbon atoms, a substituted or unsubstituted alkyl group having 1 to 10 carbon atoms, or a substituted or unsubstituted alkyl group having 1 to 6 carbon atoms. For example, the alkyl group may be a substituted or unsubstituted methyl group, a substituted or unsubstituted ethyl group, a substituted or unsubstituted isopropyl group, a substituted or unsubstituted tert-butyl group, etc., but is not limited thereto. For example, the cycloalkyl group may be, for example, a substituted or unsubstituted cyclopentyl group, a substituted or unsubstituted cyclohexyl group, a substituted or unsubstituted norbornyl group, or a substituted or unsubstituted adamantyl group, but is not limited thereto. For example, when the substituent is an aryl group, the aryl group may be a phenyl group, a biphenyl group, a terphenyl group, etc., but is not limited thereto.

According to an embodiment of the present disclosure, the compound represented by Chemical Formula 1 may have a refractive index of 1.70 or less for light in the wavelength band of 400 nm to 650 nm.

For example, the compound represented by Chemical Formula 1 may have a refractive index of 1.70 or less, 1.65 or less, or 1.60 or less for light in the wavelength band of 400 nm to 650 nm.

For example, the compound represented by Chemical Formula 1 may have a refractive index of 1.70 or less for light in the wavelength band of 460 nm, the compound represented by Chemical Formula 1 may have a refractive index of 1.65 or less for light in the wavelength band of 520 nm, and the compound represented by Chemical Formula 1 may have a refractive index of 1.60 or less for light in the wavelength band of 620 nm.

In the organic light emitting diode according to an embodiment, the capping layer may include the compound represented by Chemical Formula 1, and due to the low refractive index characteristics of the compound described above, the layer may exhibit a low refractive index. For example, the capping layer may have a refractive index of 1.70 or less for light in the wavelength band of 400 nm to 650 nm.

In an organic light emitting diode (OLED) including a capping layer having a refractive index greater than 1.70, the luminous efficiency may be lower than that of an OLED including a capping layer having a refractive index of 1.70 or less. However, although the lower limit of the refractive index of the compound of the present disclosure and of the capping layer including the compound is not specifically defined, it may be, for example, 1.50 or more, 1.51 or more, or 1.52 or more. For example, the compound represented by Chemical Formula 1 and a capping layer including the same may have a refractive index of 1.50 or more and 1.60 or less for light in the wavelength band of 400 nm to 650 nm. In addition, for example, the compound represented by Chemical Formula 1 may have a refractive index of 1.50 or more and 1.70 or less, 1.50 or more and 1.68 or less, 1.50 or more and 1.65 or less, 1.51 or more and 1.70 or less, 1.51 or more and 1.68 or less, 1.51 or more and 1.65 or less, 1.52 or more and 1.70 or less, 1.52 or more and 1.68 or less, or 1.52 or more and 1.65 or less for light in the wavelength bands of 460 nm, 520 nm, 620 nm, or 400 nm to 650 nm.

A compound according to an embodiment of the present disclosure includes a tetraphenyl structure (hereinafter, a tetraphenyl moiety (hereinafter abbreviated as TM)) wherein X is selected from nitrogen (N) or oxygen (O) and includes at least one alkyl group or cycloalkyl group connected to X. This leads to high steric hindrance in the molecular structure of the compound, reducing the packing density between compound molecules. In addition, the structure of the compound according to an embodiment may be advantageous in increasing the propagation speed of light in a medium, thereby achieving a lower refractive index. Furthermore, in the compound represented by Chemical Formula 1 of the present disclosure, R₁ and R₂ (when X is nitrogen) may be, for example, an alkyl group, a cycloalkyl group, or an aryl group substituted with an alkyl or cycloalkyl group. In this case, steric hindrance applied to all aryl structures connected to nitrogen may further lower the density, thereby achieving an even lower refractive index.

A compound according to an embodiment of the present disclosure may include a substituted or unsubstituted cycloalkyl amide structure or a substituted or unsubstituted cycloalkyl ester structure. The compound structure according to the present disclosure may have a low refractive index due to the steric hindrance and low polarizability of the cycloalkyl structure. Furthermore, the electron-withdrawing effect of the amide and ester functional groups reduces the electron density of the benzene ring, thereby enabling a low refractive index when used to form as a capping layer.

When a compound according to an embodiment of the present disclosure includes a substituted or unsubstituted cycloalkyl amide structure, it may be a secondary amide where R₂ is hydrogen, or a tertiary amide where R₂ is a substituted or unsubstituted alkyl group or a substituted or unsubstituted cycloalkyl group. Additionally, in a compound according to an embodiment of the present disclosure, when R₂ is hydrogen, the intermolecular bonding strength may be strengthened through hydrogen bonding, resulting in excellent adhesion to the cathode material formed as a capping layer. Additionally, in a compound according to an embodiment of the present disclosure, when R₂ is not hydrogen but is a substituted or unsubstituted alkyl or a substituted or unsubstituted cycloalkyl, R₂ may cause additional steric hindrance into the compound, resulting in a low refractive index when used to form as a capping layer.

In a compound according to an embodiment of the present disclosure, the steric structure and size of the molecule may be controlled by selecting the number of alkyl or cycloalkyl groups to be introduced. Through this, the packing density of a thin film formed from the compound and the crystallinity of the compound may be controlled. However, although lowering the packing density of the thin film may reduce the refractive index, low crystallinity may cause a difference in the deposition temperature with other materials during the manufacturing process of an organic light emitting diode, thereby reducing the productivity of the diode. In addition, due to a low glass transition temperature, the thermal stability of the diode may also be reduced when undergoing other processes. Accordingly, the alkyl or cycloalkyl groups introduced into the compound need to be selected within an appropriate structural and number range.

For example, in a compound according to an embodiment of the present disclosure, the number of alkyl or cycloalkyl groups introduced into the compound may be 4 to 7, 4 to 8, or 4 to 9. In this case, both low refractive index and thermal stability during the manufacturing processes may be satisfied.

In addition, in the molecular structure of a compound according to an embodiment of the present disclosure, alkyl or cycloalkyl groups may be introduced in all structures connected to nitrogen. In this case, both low refractive index and thermal stability during the manufacturing process may be satisfied.

A compound according to an embodiment of the present disclosure and a capping layer including the same may have a light transmittance of about 80% or more (an absorption coefficient (K) of 0.02) in the visible light region with wavelength ranging from 400 nm to 460 nm. As a result, light loss from the diode may be reduced, thereby improving luminous efficiency and external quantum efficiency of the organic light emitting diode. A compound according to an embodiment and a capping layer including the same may have a light transmittance of 82% or more, 84% or more, 86% or more, 88% or more, 89% or more, 90% or more, 91% or more, 92% or more, 93% or more, 94% or more, 95% or more, or 96% or more in the visible light region with wavelength ranging from 400 nm to 460 nm.

For example, a compound according to an embodiment of the present disclosure and a capping layer including the same may have a light transmittance of 80% or more in the visible light region with wavelength ranging from 400 nm to 410 nm.

In an organic light emitting diode according to an embodiment of the present disclosure, the capping layer exhibiting low-refractive-index characteristics serves as the final layer through which light generated from the diode passes. When absorption occurs in the visible light region with wavelength ranging from 400 nm to 410 nm, the efficiency of the diode may be reduced.

In the case of a capping layer compound according to prior art, absorption may occur in the wavelength band of 400 nm to 410 nm, which is the deep blue region. However, in the case of a compound according to an embodiment and a capping layer including the same, the light transmittance is 80% or more in the wavelength band of 400 nm to 410 nm. Therefore, light loss from the diode may be minimized and the diode efficiency may be significantly improved.

An organic compound according to an embodiment of the present disclosure may maintain a wide bandgap that does not allow for the absorption of light in the visible wavelength band. Therefore, a low refractive index may be maintained.

In addition, a compound (represented by Chemical Formula 1) according to an embodiment of the present disclosure may absorb high-energy wavelengths with a wavelength of less than about 400 nm. Therefore, a capping layer including the compound represented by Chemical Formula 1 may minimize damage to organic materials within an organic light emitting diode.

Furthermore, a thin film including a compound according to an embodiment of the present disclosure may exhibit excellent thin film alignment and thus high stability.

Additionally, a compound according to an embodiment of the present disclosure has appropriate Tg and Td, thereby suppressing intermolecular recrystallization during the manufacturing process of an organic light emitting diode. Therefore, an organic light emitting diode including a capping layer according to an embodiment may exhibit excellent color purity and significantly improved external luminous efficiency.

A capping layer including a compound according to an embodiment of the present disclosure may be disposed as a single layer or as multiple layers on the surface of the first electrode or the second electrode of the organic light emitting diode. For example, two capping layers may be disposed on one surface of the second electrode.

For example, when an organic light emitting diode includes a plurality of capping layers, at least one of the plurality of capping layers may include one or more compounds selected from the compounds represented by Chemical Formula 1. For example, in an organic light emitting diode including a double-layer capping layer structure, a capping layer (a first capping layer) disposed on an electrode and in contact with the electrode includes a compound represented by Chemical Formula 1, and a capping layer (a second capping layer) disposed on the first capping layer may include a compound represented by Chemical Formula 1, and a material different from the compound represented by Chemical Formula 1.

Here, the material different from the compound represented by Chemical Formula 1 is not particularly limited and may be any material commonly used as a capping layer compound. Non-limiting examples of the other materials include an arylamine derivative, a naphthalene derivative, an anthracene derivative, a phenanthrene derivative, a carbazole derivative, a pyridine derivative, a dibenzofuran derivative, a dibenzothiophene derivative, a pyrimidine derivative, a quinoline derivative, an isoquinoline derivative, a benzoxazole derivative, a benzothiazole derivative, a benzimidazole derivative, N4,N4′-bis[4-[bis(3-methylphenyl)amino]phenyl]-N4,N4′-diphenyl-[1,1′-biphenyl]-4,4′-diamine (DNTPD), tris(8-hydroxyquinolinato)aluminum (Alq₃), LiF, Liq, Li₂O, BaO, NaCl, or CsF.

In a structure in which the capping layer according to an embodiment includes a plurality of capping layers, the refractive indices of the respective capping layers may differ from each other. For example, the difference in refractive indices between the first capping layer material and the second capping layer material may be utilized to further improve the luminous efficiency of the organic light emitting diode.

According to an embodiment of the present disclosure, the organic compound represented by Chemical Formula 1 may be represented by Chemical Formulae 2-1 to 2-6 and 3-1 to 3-6 below.

In the following Chemical Formulas, R₁ and R₂, and their substituents are as defined in Chemical Formula 1.

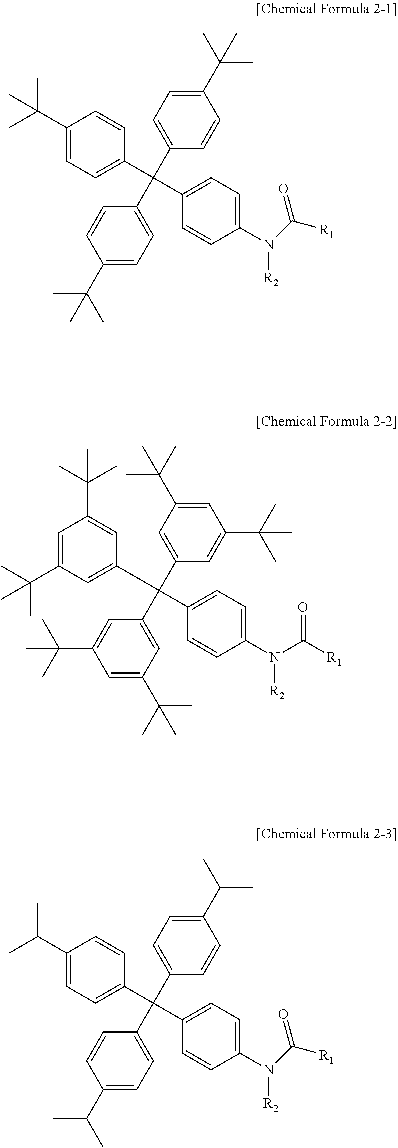

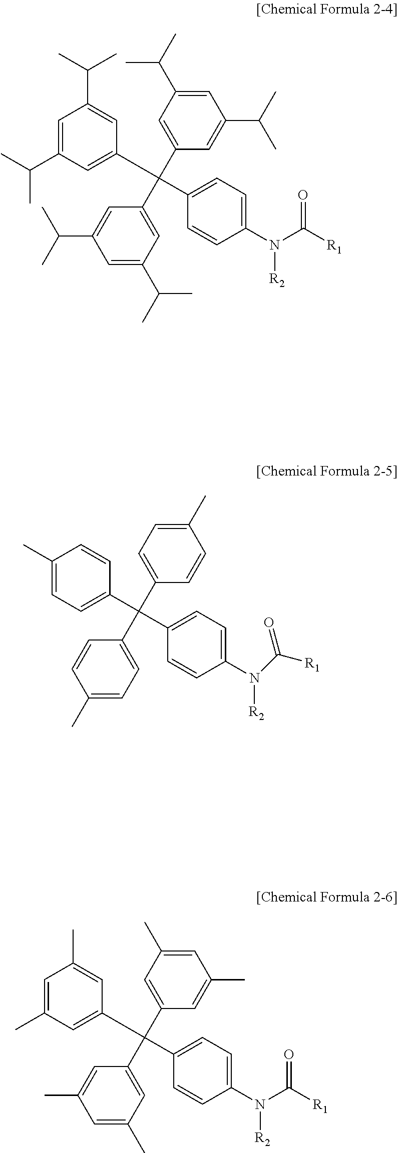

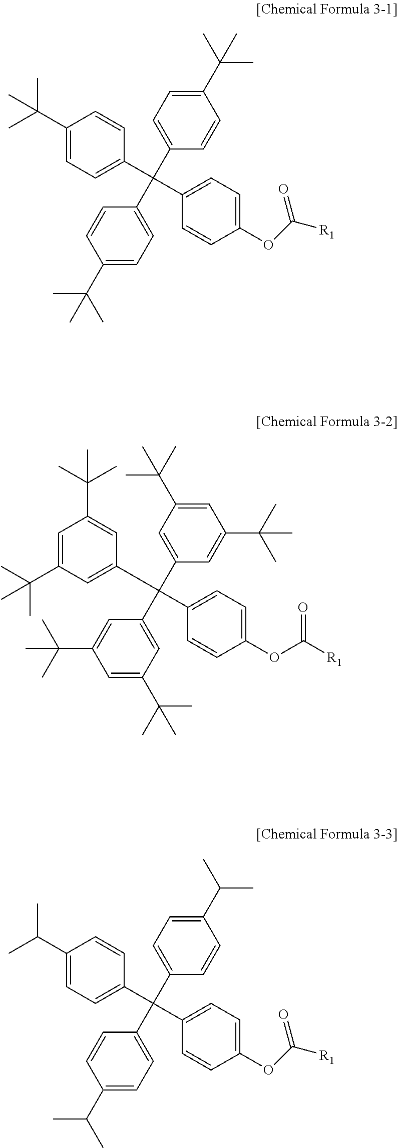

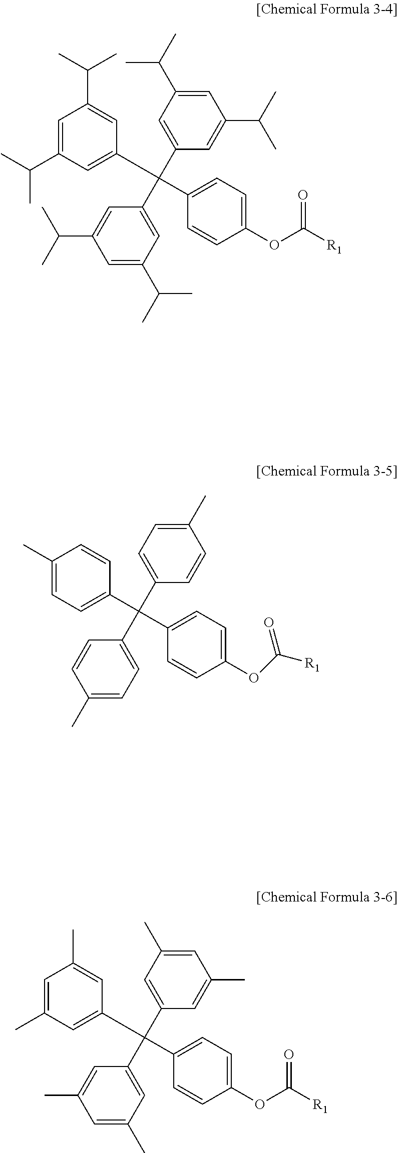

According to an embodiment of the present disclosure, the tetraphenyl moiety (hereinafter abbreviated as TM), which is a part of the structures of Chemical Formula 1, Chemical Formulae 2-1 to 2-6, and Chemical Formulae 3-1 to 3-6, may be selected from the structures TM1 to TM6 below. (In the structures below, * indicates a position at which the structure is bonded to the Chemical Formula via a single bond.)

### [Tetraphenyl Moiety (Hereinafter Abbreviated as TM)]

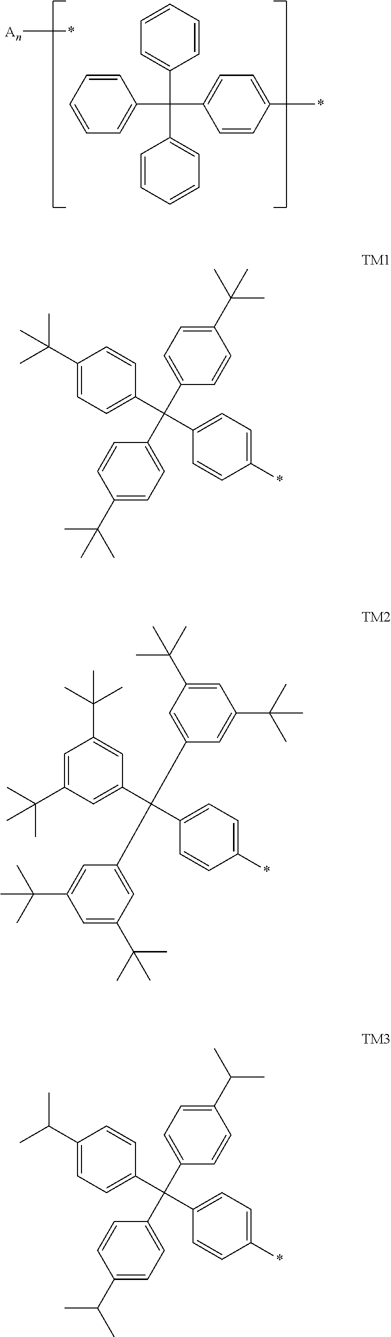

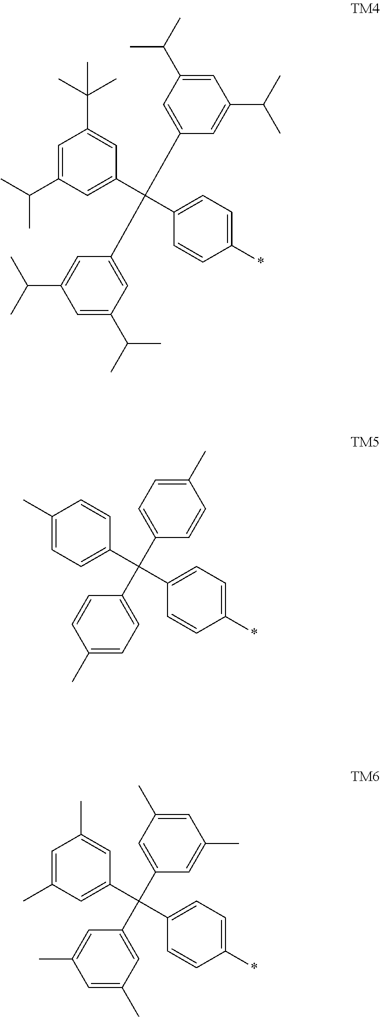

According to an example of the present disclosure, A, R₁, and R₂ in Chemical Formula 1, Chemical Formulae 2-1 to 2-6, and Chemical Formulae 3-1 to 3-6 may be selected from hydrogen and the structures F1 to F61 shown below. (In the structures below, * indicates a portion at which the structure is bonded to the Chemical Formula via a single bond.)

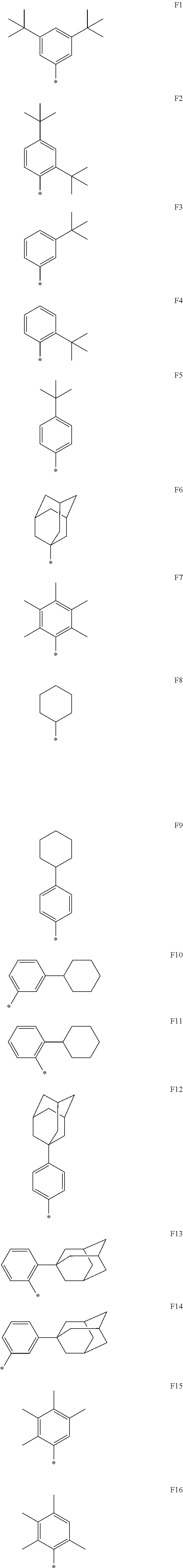

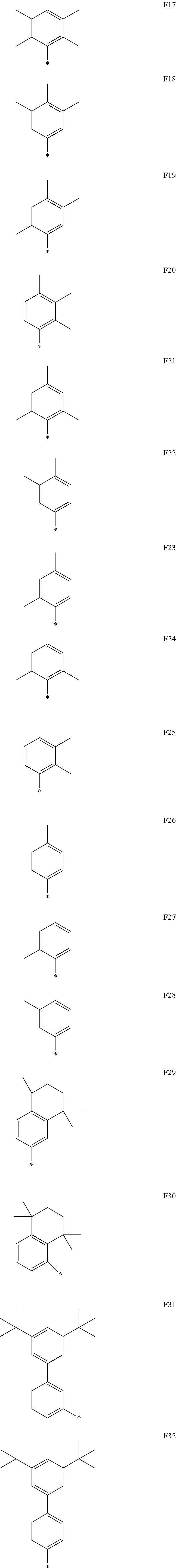

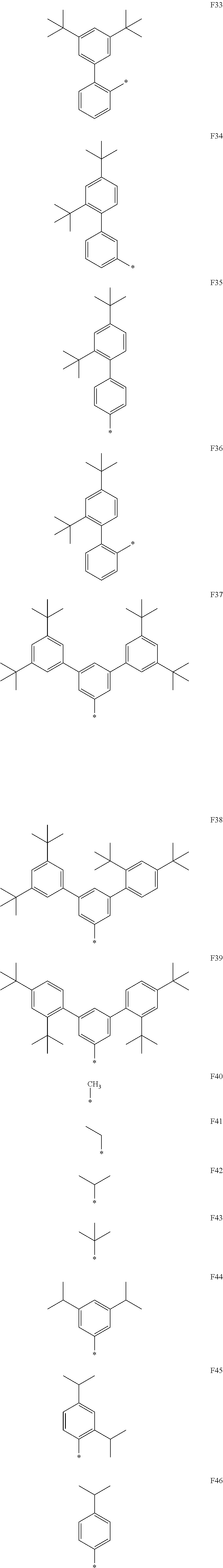

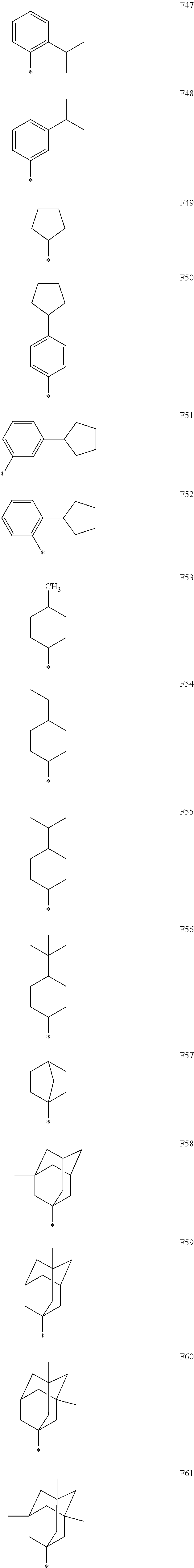

According to an embodiment of the present disclosure, the compound represented by Chemical Formula 1 may be selected from the following compounds, but is not limited thereto.

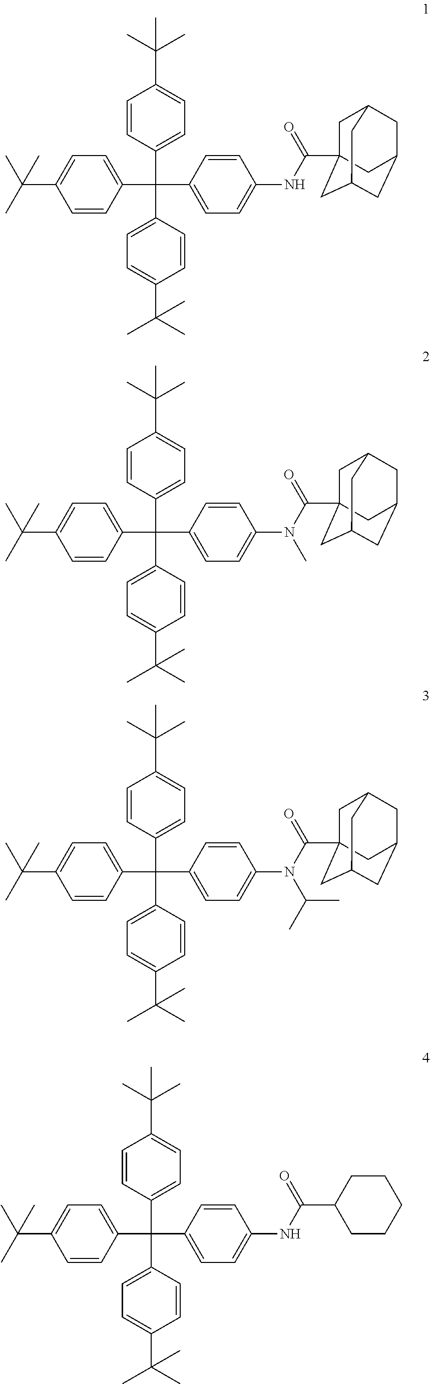

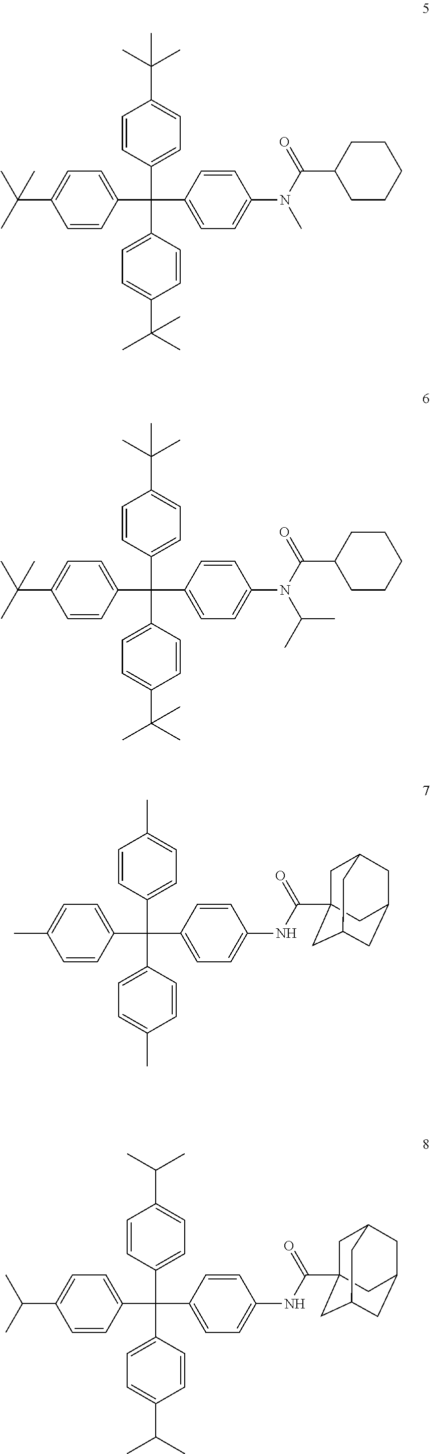 [SMILES: CN(C(=O)C1CCCCC1)c1ccc(C(c2ccc(C(C)(C)C)cc2)(c2ccc(C(C)(C)C)cc2)c2ccc(C(C)(C)C)cc2)cc1 ; CC(C)N(C(=O)C1CCCCC1)c1ccc(C(c2ccc(C(C)(C)C)cc2)(c2ccc(C(C)(C)C)cc2)c2ccc(C(C)(C)C)cc2)cc1 ; Cc1ccc(C(c2ccc(C)cc2)(c2ccc(C)cc2)c2ccc(NC(=O)C34CC5CC(CC(C5)C3)C4)cc2)cc1 ; CC(C)c1ccc(C(c2ccc(NC(=O)C34CC5CC(CC(C5)C3)C4)cc2)(c2ccc(C(C)C)cc2)c2ccc(C(C)C)cc2)cc1]

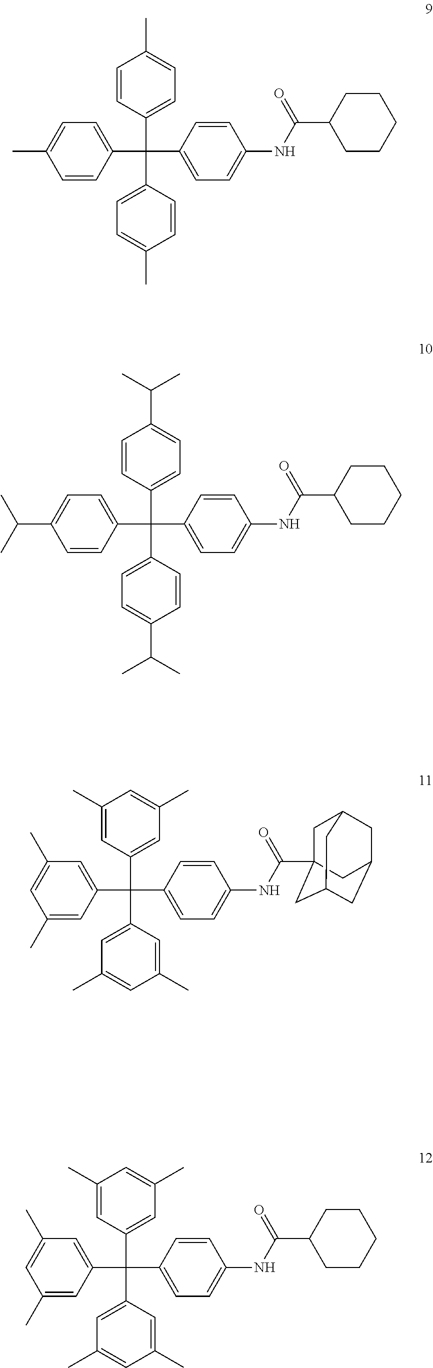 [SMILES: Cc1ccc(C(c2ccc(C)cc2)(c2ccc(C)cc2)c2ccc(NC(=O)C3CCCCC3)cc2)cc1 ; CC(C)c1ccc(C(c2ccc(NC(=O)C3CCCCC3)cc2)(c2ccc(C(C)C)cc2)c2ccc(C(C)C)cc2)cc1 ; Cc1cc(C)cc(C(c2ccc(NC(=O)C34CC5CC(CC(C5)C3)C4)cc2)(c2cc(C)cc(C)c2)c2cc(C)cc(C)c2)c1 ; Cc1cc(C)cc(C(c2ccc(NC(=O)C3CCCCC3)cc2)(c2cc(C)cc(C)c2)c2cc(C)cc(C)c2)c1]

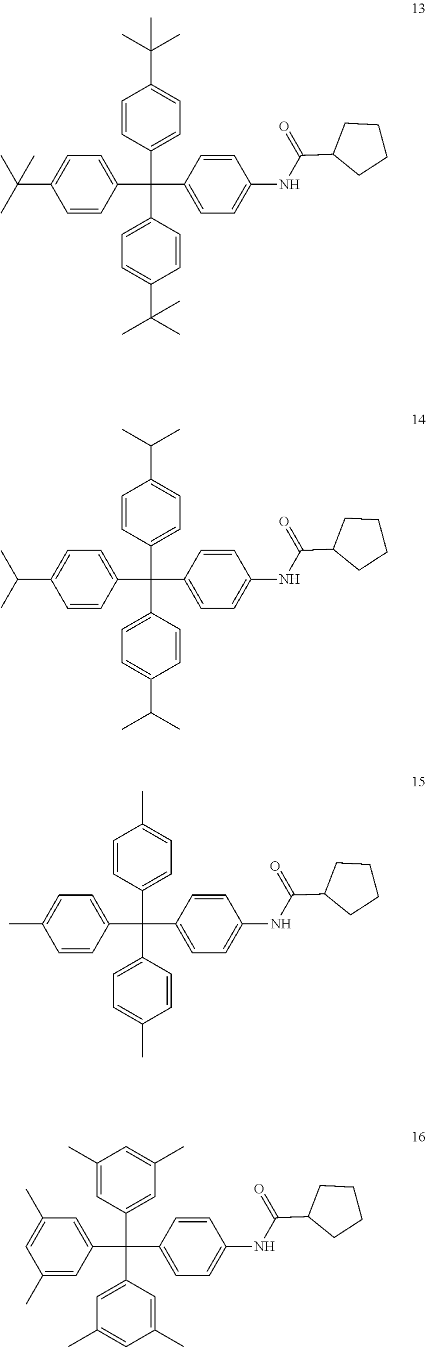 [SMILES: CC(C)(C)c1ccc(C(c2ccc(NC(=O)C3CCCC3)cc2)(c2ccc(C(C)(C)C)cc2)c2ccc(C(C)(C)C)cc2)cc1 ; CC(C)c1ccc(C(c2ccc(NC(=O)C3CCCC3)cc2)(c2ccc(C(C)C)cc2)c2ccc(C(C)C)cc2)cc1 ; Cc1ccc(C(c2ccc(C)cc2)(c2ccc(C)cc2)c2ccc(NC(=O)C3CCCC3)cc2)cc1 ; Cc1cc(C)cc(C(c2ccc(NC(=O)C3CCCC3)cc2)(c2cc(C)cc(C)c2)c2cc(C)cc(C)c2)c1]

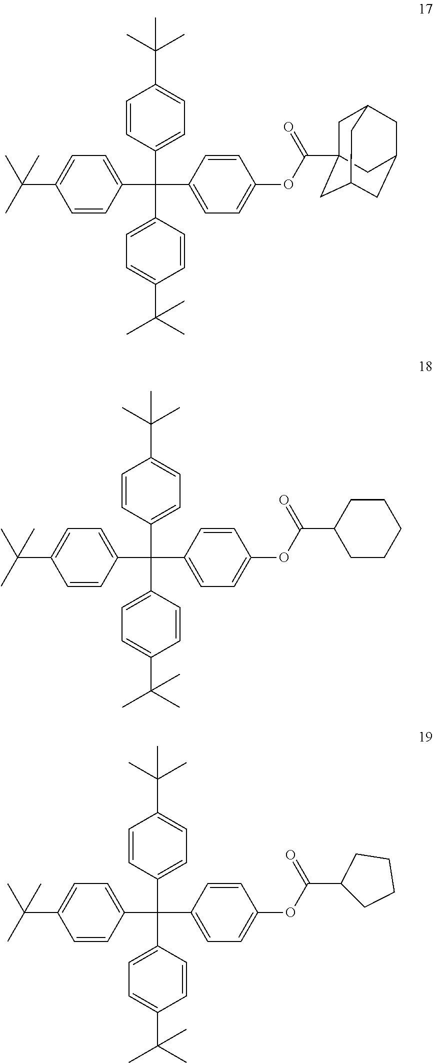

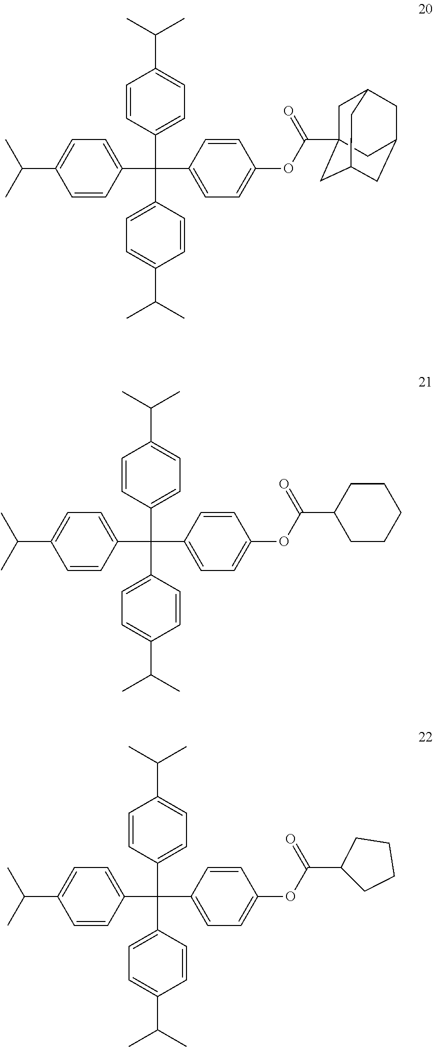 [SMILES: CC(C)c1ccc(C(c2ccc(OC(=O)C34CC5CC(CC(C5)C3)C4)cc2)(c2ccc(C(C)C)cc2)c2ccc(C(C)C)cc2)cc1 ; CC(C)c1ccc(C(c2ccc(OC(=O)C3CCCCC3)cc2)(c2ccc(C(C)C)cc2)c2ccc(C(C)C)cc2)cc1 ; CC(C)c1ccc(C(c2ccc(OC(=O)C3CCCC3)cc2)(c2ccc(C(C)C)cc2)c2ccc(C(C)C)cc2)cc1]

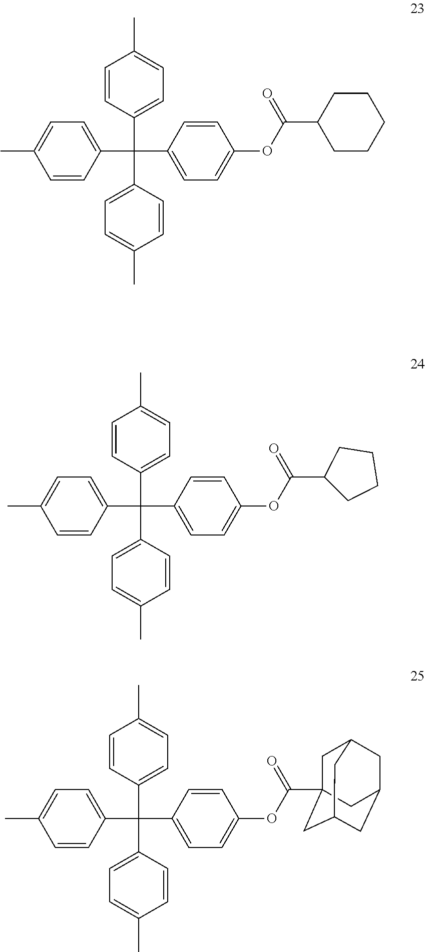

Compounds 1 to 25 may be represented as shown in Table 1 below.

TABLE 1

| Compound | Chemical | | |
|---|---|---|---|
| NO. | structure | R₁ | R₂ |
| 1 | 2-1 | F6 | H |
| 2 | 2-1 | F6 | F40 |
| 3 | 2-1 | F6 | F42 |
| 4 | 2-1 | F8 | H |
| 5 | 2-1 | F8 | F40 |
| 6 | 2-1 | F8 | F42 |
| 7 | 2-5 | F6 | H |
| 8 | 2-3 | F6 | H |
| 9 | 2-5 | F8 | H |
| 10 | 2-3 | F8 | H |
| 11 | 2-6 | F6 | H |
| 12 | 2-6 | F8 | H |
| 13 | 2-1 | F49 | H |
| 14 | 2-3 | F49 | H |
| 15 | 2-5 | F49 | H |
| 16 | 2-6 | F49 | H |
| 17 | 3-1 | F6 | — |
| 18 | 3-1 | F8 | — |
| 19 | 3-1 | F49 | — |
| 20 | 3-3 | F6 | — |
| 21 | 3-3 | F8 | — |
| 22 | 3-3 | F49 | — |
| 23 | 3-5 | F8 | — |
| 24 | 3-5 | F49 | — |
| 25 | 3-5 | F6 | — |

The compound represented by Chemical Formula 1 according to the present disclosure may be selected from, but is not limited to, the group consisting of compounds represented by the following Compounds 26 to 352. Compounds 26 to 352 may be represented as shown in Tables 2 to 5 below.

TABLE 2

| Compound | Chemical | | |
|---|---|---|---|
| NO. | structure | R₁ | R₂ |
| 26 | 2-1 | F56 | H |
| 27 | 2-1 | F53 | H |
| 28 | 2-1 | F54 | H |
| 29 | 2-1 | F55 | H |
| 30 | 2-1 | F58 | H |
| 31 | 2-1 | F60 | H |
| 32 | 2-1 | F61 | H |
| 33 | 2-1 | F49 | F40 |
| 34 | 2-1 | F56 | F40 |
| 35 | 2-1 | F53 | F40 |
| 36 | 2-1 | F54 | F40 |
| 37 | 2-1 | F55 | F40 |
| 38 | 2-1 | F58 | F40 |
| 39 | 2-1 | F60 | F40 |
| 40 | 2-1 | F61 | F40 |
| 41 | 2-1 | F49 | F42 |
| 42 | 2-1 | F56 | F42 |
| 43 | 2-1 | F53 | F42 |
| 44 | 2-1 | F54 | F42 |
| 45 | 2-1 | F55 | F42 |
| 46 | 2-1 | F58 | F42 |
| 47 | 2-1 | F60 | F42 |
| 48 | 2-1 | F61 | F42 |
| 49 | 2-1 | F6 | F43 |
| 50 | 2-1 | F8 | F43 |
| 51 | 2-1 | F49 | F43 |
| 52 | 2-1 | F56 | F43 |
| 53 | 2-1 | F53 | F43 |
| 54 | 2-1 | F54 | F43 |
| 55 | 2-1 | F55 | F43 |
| 56 | 2-1 | F58 | F43 |
| 57 | 2-1 | F60 | F43 |
| 58 | 2-1 | F61 | F43 |
| 59 | 2-3 | F56 | H |
| 60 | 2-3 | F53 | H |
| 61 | 2-3 | F54 | H |
| 62 | 2-3 | F55 | H |
| 63 | 2-3 | F58 | H |
| 64 | 2-3 | F60 | H |
| 65 | 2-3 | F61 | H |
| 66 | 2-3 | F6 | F40 |
| 67 | 2-3 | F8 | F40 |
| 68 | 2-3 | F49 | F40 |
| 69 | 2-3 | F56 | F40 |
| 70 | 2-3 | F53 | F40 |
| 71 | 2-3 | F54 | F40 |
| 72 | 2-3 | F55 | F40 |
| 73 | 2-3 | F58 | F40 |
| 74 | 2-3 | F60 | F40 |
| 75 | 2-3 | F61 | F40 |
| 76 | 2-3 | F6 | F42 |
| 77 | 2-3 | F8 | F42 |
| 78 | 2-3 | F49 | F42 |
| 79 | 2-3 | F56 | F42 |
| 80 | 2-3 | F53 | F42 |
| 81 | 2-3 | F54 | F42 |
| 82 | 2-3 | F55 | F42 |
| 83 | 2-3 | F58 | F42 |
| 84 | 2-3 | F60 | F42 |
| 85 | 2-3 | F61 | F42 |
| 86 | 2-3 | F6 | F43 |
| 87 | 2-3 | F8 | F43 |
| 88 | 2-3 | F49 | F43 |
| 89 | 2-3 | F56 | F43 |
| 90 | 2-3 | F53 | F43 |
| 91 | 2-3 | F54 | F43 |
| 92 | 2-3 | F55 | F43 |
| 93 | 2-3 | F58 | F43 |
| 94 | 2-3 | F60 | F43 |
| 95 | 2-3 | F61 | F43 |
| 96 | 2-5 | F56 | H |
| 97 | 2-5 | F53 | H |
| 98 | 2-5 | F54 | H |
| 99 | 2-5 | F55 | H |
| 100 | 2-5 | F58 | H |
| 101 | 2-5 | F60 | H |
| 102 | 2-5 | F61 | H |
| 103 | 2-5 | F6 | F40 |
| 104 | 2-5 | F8 | F40 |
| 105 | 2-5 | F49 | F40 |
| 106 | 2-5 | F56 | F40 |
| 107 | 2-5 | F53 | F40 |
| 108 | 2-5 | F54 | F40 |
| 109 | 2-5 | F55 | F40 |
| 110 | 2-5 | F58 | F40 |
| 111 | 2-5 | F60 | F40 |
| 112 | 2-5 | F61 | F40 |
| 113 | 2-5 | F6 | F42 |
| 114 | 2-5 | F8 | F42 |
| 115 | 2-5 | F49 | F42 |
| 116 | 2-5 | F56 | F42 |
| 117 | 2-5 | F53 | F42 |
| 118 | 2-5 | F54 | F42 |
| 119 | 2-5 | F55 | F42 |
| 120 | 2-5 | F58 | F42 |
| 121 | 2-5 | F60 | F42 |
| 122 | 2-5 | F61 | F42 |
| 123 | 2-5 | F6 | F43 |
| 124 | 2-5 | F8 | F43 |
| 125 | 2-5 | F49 | F43 |
| 126 | 2-5 | F56 | F43 |
| 127 | 2-5 | F53 | F43 |
| 128 | 2-5 | F54 | F43 |
| 129 | 2-5 | F55 | F43 |
| 130 | 2-5 | F58 | F43 |

TABLE 3

| Compound | Chemical | | |
|---|---|---|---|
| NO. | structure | R₁ | R₂ |
| 131 | 2-5 | F60 | F43 |
| 132 | 2-5 | F61 | F43 |
| 133 | 2-2 | F6 | H |
| 134 | 2-2 | F8 | H |
| 135 | 2-2 | F49 | H |
| 136 | 2-2 | F56 | H |
| 137 | 2-2 | F53 | H |
| 138 | 2-2 | F54 | H |
| 139 | 2-2 | F55 | H |
| 140 | 2-2 | F58 | H |
| 141 | 2-2 | F60 | H |
| 142 | 2-2 | F61 | H |
| 143 | 2-2 | F6 | F40 |
| 144 | 2-2 | F8 | F40 |
| 145 | 2-2 | F49 | F40 |
| 146 | 2-2 | F56 | F40 |
| 147 | 2-2 | F53 | F40 |
| 148 | 2-2 | F54 | F40 |
| 149 | 2-2 | F55 | F40 |
| 150 | 2-2 | F58 | F40 |
| 151 | 2-2 | F60 | F40 |
| 152 | 2-2 | F61 | F40 |
| 153 | 2-2 | F6 | F42 |
| 154 | 2-2 | F8 | F42 |
| 155 | 2-2 | F49 | F42 |
| 156 | 2-2 | F56 | F42 |
| 157 | 2-2 | F53 | F42 |
| 158 | 2-2 | F54 | F42 |
| 159 | 2-2 | F55 | F42 |
| 160 | 2-2 | F58 | F42 |
| 161 | 2-2 | F60 | F42 |
| 162 | 2-2 | F61 | F42 |
| 163 | 2-2 | F6 | F43 |
| 164 | 2-2 | F8 | F43 |
| 165 | 2-2 | F49 | F43 |
| 166 | 2-2 | F56 | F43 |
| 167 | 2-2 | F53 | F43 |
| 168 | 2-2 | F54 | F43 |
| 169 | 2-2 | F55 | F43 |
| 170 | 2-2 | F58 | F43 |
| 171 | 2-2 | F60 | F43 |
| 172 | 2-2 | F61 | F43 |
| 173 | 2-4 | F6 | H |
| 174 | 2-4 | F8 | H |
| 175 | 2-4 | F49 | H |
| 176 | 2-4 | F56 | H |
| 177 | 2-4 | F53 | H |
| 178 | 2-4 | F54 | H |
| 179 | 2-4 | F55 | H |
| 180 | 2-4 | F58 | H |
| 181 | 2-4 | F60 | H |
| 182 | 2-4 | F61 | H |
| 183 | 2-4 | F6 | F40 |
| 184 | 2-4 | F8 | F40 |
| 185 | 2-4 | F49 | F40 |
| 186 | 2-4 | F56 | F40 |
| 187 | 2-4 | F53 | F40 |
| 188 | 2-4 | F54 | F40 |
| 189 | 2-4 | F55 | F40 |
| 190 | 2-4 | F58 | F40 |
| 191 | 2-4 | F60 | F40 |
| 192 | 2-4 | F61 | F40 |
| 193 | 2-4 | F6 | F42 |
| 194 | 2-4 | F8 | F42 |
| 195 | 2-4 | F49 | F42 |
| 196 | 2-4 | F56 | F42 |
| 197 | 2-4 | F53 | F42 |
| 198 | 2-4 | F54 | F42 |
| 199 | 2-4 | F55 | F42 |
| 200 | 2-4 | F58 | F42 |
| 201 | 2-4 | F60 | F42 |
| 202 | 2-4 | F61 | F42 |
| 203 | 2-4 | F6 | F43 |
| 204 | 2-4 | F8 | F43 |
| 205 | 2-4 | F49 | F43 |
| 206 | 2-4 | F56 | F43 |
| 207 | 2-4 | F53 | F43 |
| 208 | 2-4 | F54 | F43 |
| 209 | 2-4 | F55 | F43 |
| 210 | 2-4 | F58 | F43 |
| 211 | 2-4 | F60 | F43 |
| 212 | 2-4 | F61 | F43 |
| 213 | 2-6 | F6 | H |
| 214 | 2-6 | F8 | H |
| 215 | 2-6 | F56 | H |
| 216 | 2-6 | F53 | H |
| 217 | 2-6 | F54 | H |
| 218 | 2-6 | F55 | H |
| 219 | 2-6 | F58 | H |
| 220 | 2-6 | F60 | H |
| 221 | 2-6 | F61 | H |
| 222 | 2-6 | F6 | F40 |
| 223 | 2-6 | F8 | F40 |
| 224 | 2-6 | F49 | F40 |
| 225 | 2-6 | F56 | F40 |
| 226 | 2-6 | F53 | F40 |
| 227 | 2-6 | F54 | F40 |
| 228 | 2-6 | F55 | F40 |
| 229 | 2-6 | F58 | F40 |
| 230 | 2-6 | F60 | F40 |
| 231 | 2-6 | F61 | F40 |
| 232 | 2-6 | F6 | F42 |
| 233 | 2-6 | F8 | F42 |
| 234 | 2-6 | F49 | F42 |
| 235 | 2-6 | F56 | F42 |

TABLE 4

| Compound | Chemical | | |
|---|---|---|---|
| NO. | structure | R₁ | R₂ |
| 236 | 2-6 | F53 | F42 |
| 237 | 2-6 | F54 | F42 |
| 238 | 2-6 | F55 | F42 |
| 239 | 2-6 | F58 | F42 |
| 240 | 2-6 | F60 | F42 |
| 241 | 2-6 | F61 | F42 |
| 242 | 2-6 | F6 | F43 |
| 243 | 2-6 | F8 | F43 |
| 244 | 2-6 | F49 | F43 |
| 245 | 2-6 | F56 | F43 |
| 246 | 2-6 | F53 | F43 |
| 247 | 2-6 | F54 | F43 |
| 248 | 2-6 | F55 | F43 |
| 249 | 2-6 | F58 | F43 |
| 250 | 2-6 | F60 | F43 |
| 251 | 2-6 | F61 | F43 |
| 252 | 3-1 | F56 | — |
| 253 | 3-1 | F53 | — |
| 254 | 3-1 | F54 | — |
| 255 | 3-1 | F55 | — |
| 256 | 3-1 | F58 | — |
| 257 | 3-1 | F60 | — |
| 258 | 3-1 | F61 | — |
| 259 | 3-3 | F56 | — |
| 260 | 3-3 | F53 | — |
| 261 | 3-3 | F54 | — |
| 262 | 3-3 | F55 | — |
| 263 | 3-3 | F58 | — |
| 264 | 3-3 | F60 | — |
| 265 | 3-3 | F61 | — |
| 266 | 3-5 | F56 | — |
| 267 | 3-5 | F53 | — |
| 268 | 3-5 | F54 | — |
| 269 | 3-5 | F55 | — |
| 270 | 3-5 | F58 | — |
| 271 | 3-5 | F60 | — |
| 272 | 3-5 | F61 | — |
| 273 | 3-2 | F6 | — |
| 274 | 3-2 | F8 | — |
| 275 | 3-2 | F49 | — |
| 276 | 3-2 | F56 | — |
| 277 | 3-2 | F53 | — |
| 278 | 3-2 | F54 | — |
| 279 | 3-2 | F55 | — |
| 280 | 3-2 | F58 | — |
| 281 | 3-2 | F60 | — |
| 282 | 3-2 | F61 | — |
| 283 | 3-4 | F6 | — |
| 284 | 3-4 | F8 | — |
| 285 | 3-4 | F49 | — |
| 286 | 3-4 | F56 | — |
| 287 | 3-4 | F53 | — |
| 288 | 3-4 | F54 | — |
| 289 | 3-4 | F55 | — |
| 290 | 3-4 | F58 | — |
| 291 | 3-4 | F60 | — |
| 292 | 3-4 | F61 | — |
| 293 | 3-6 | F6 | — |
| 294 | 3-6 | F8 | — |
| 295 | 3-6 | F49 | — |
| 296 | 3-6 | F56 | — |
| 297 | 3-6 | F53 | — |
| 298 | 3-6 | F54 | — |
| 299 | 3-6 | F55 | — |
| 300 | 3-6 | F58 | — |
| 301 | 3-6 | F60 | — |
| 302 | 3-6 | F61 | — |
| 303 | 2-1 | F1 | H |
| 304 | 2-1 | F2 | H |
| 305 | 2-1 | F3 | H |
| 306 | 2-1 | F4 | H |
| 307 | 2-1 | F5 | H |
| 308 | 2-1 | F7 | H |
| 309 | 2-1 | F10 | H |
| 310 | 2-1 | F11 | H |
| 311 | 2-1 | F12 | H |
| 312 | 2-1 | F13 | H |
| 313 | 2-1 | F14 | H |
| 314 | 2-1 | F15 | H |
| 315 | 2-1 | F16 | H |
| 316 | 2-1 | F17 | H |
| 317 | 2-1 | F18 | H |
| 318 | 2-1 | F19 | H |
| 319 | 2-1 | F20 | H |
| 320 | 2-1 | F21 | H |
| 321 | 2-1 | F22 | H |
| 322 | 2-1 | F23 | H |
| 323 | 2-1 | F24 | H |
| 324 | 2-1 | F25 | H |
| 325 | 2-1 | F26 | H |
| 326 | 2-1 | F27 | H |
| 327 | 2-1 | F28 | H |
| 328 | 2-1 | F29 | H |
| 329 | 2-1 | F30 | H |
| 330 | 2-1 | F31 | H |
| 331 | 2-1 | F32 | H |
| 332 | 2-1 | F33 | H |
| 333 | 2-1 | F34 | H |
| 334 | 2-1 | F35 | H |
| 335 | 2-1 | F36 | H |
| 336 | 2-1 | F37 | H |
| 337 | 2-1 | F38 | H |
| 338 | 2-1 | F39 | H |
| 339 | 2-1 | F40 | H |
| 340 | 2-1 | F41 | H |

TABLE 5

| Compound | Chemical | | |
|---|---|---|---|
| NO. | structure | R₁ | R₂ |
| 341 | 2-1 | F42 | H |
| 342 | 2-1 | F43 | H |
| 343 | 2-1 | F44 | H |
| 344 | 2-1 | F45 | H |
| 345 | 2-1 | F46 | H |
| 346 | 2-1 | F47 | H |
| 347 | 2-1 | F48 | H |
| 348 | 2-1 | F50 | H |
| 349 | 2-1 | F51 | H |
| 350 | 2-1 | F52 | H |
| 351 | 2-1 | F56 | H |
| 352 | 2-1 | F59 | H |

The compound represented by Chemical Formula 1 may be a compound substituted with a halogen such as fluorine, and may be selected from the group consisting of the compounds represented by 353 to 360, but is not limited thereto.

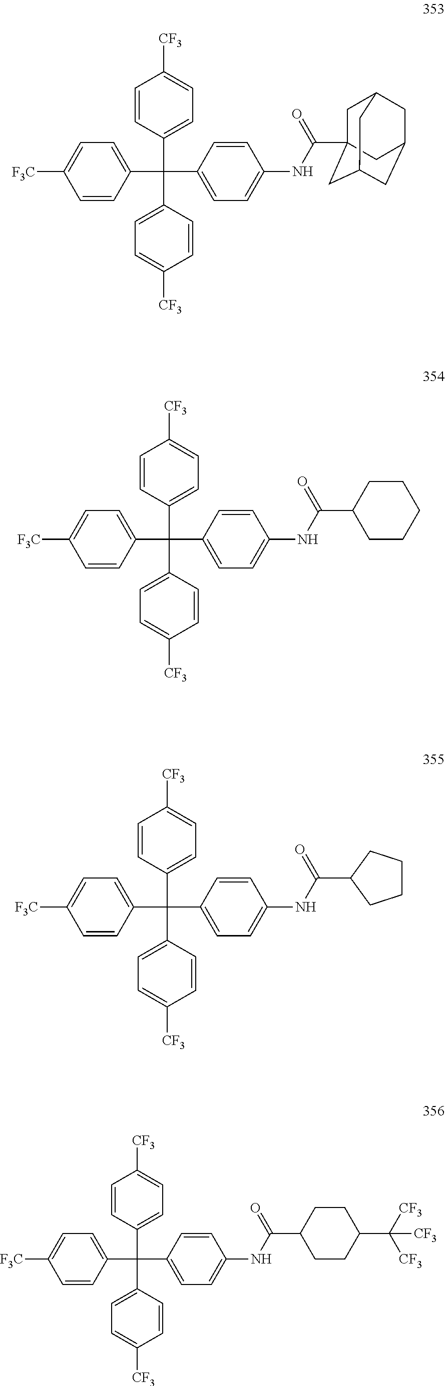 [SMILES: O=C(Nc1ccc(C(c2ccc(C(F)(F)F)cc2)(c2ccc(C(F)(F)F)cc2)c2ccc(C(F)(F)F)cc2)cc1)C12CC3CC(CC(C3)C1)C2 ; O=C(Nc1ccc(C(c2ccc(C(F)(F)F)cc2)(c2ccc(C(F)(F)F)cc2)c2ccc(C(F)(F)F)cc2)cc1)C1CCCCC1 ; O=C(Nc1ccc(C(c2ccc(C(F)(F)F)cc2)(c2ccc(C(F)(F)F)cc2)c2ccc(C(F)(F)F)cc2)cc1)C1CCCC1 ; O=C(Nc1ccc(C(c2ccc(C(F)(F)F)cc2)(c2ccc(C(F)(F)F)cc2)c2ccc(C(F)(F)F)cc2)cc1)C1CCC(C(C(F)(F)F)(C(F)(F)F)C(F)(F)F)CC1]

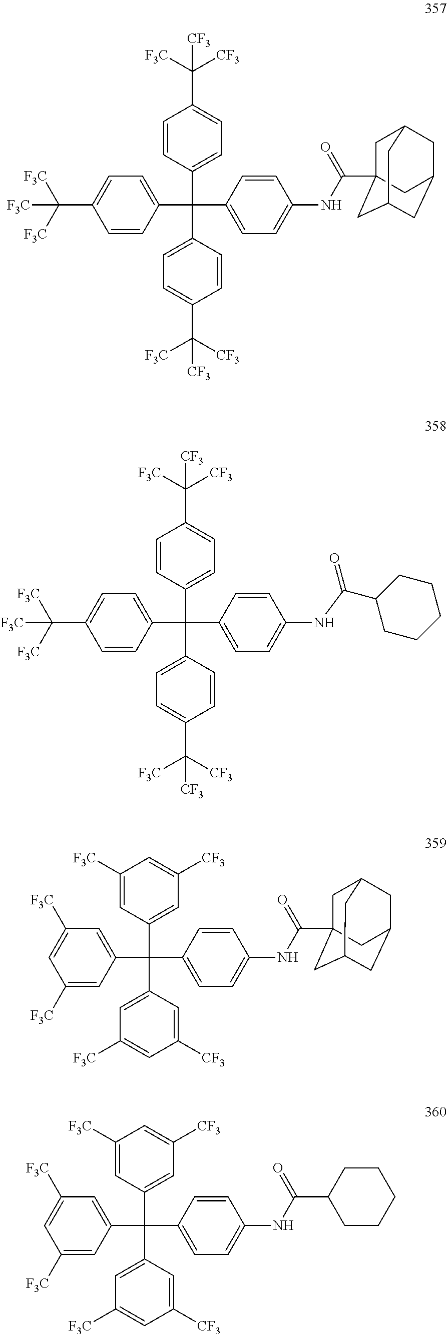

As described above, the organic light emitting diode according to an embodiment may include a first electrode, a second electrode facing the first electrode, one or more organic layers disposed between the first electrode and second electrode, and a capping layer disposed on an outer side of one or more of the first electrode and second electrode, wherein the capping layer may include a compound represented by Chemical Formula 1.

The organic layer may include one or more of a hole injection layer, a hole transport layer, an electron blocking layer, an emitting layer, a hole blocking layer, an electron transport layer, and an electron injection layer, and may further include a charge generation layer, a hole transport auxiliary layer, an emission auxiliary layer, an electron transport auxiliary layer, etc.

For example, the organic light emitting diode may have a structure in which a first electrode (anode), a hole injection layer (HIL), a hole transport layer (HTL), an electron blocking layer (EBL), an emitting layer (EML), a hole blocking layer (HBL), an electron transport layer (ETL), an electron injection layer (EIL), and a second electrode (cathode) are sequentially stacked.

For example, the first electrode may include a material such as indium tin oxide (ITO), indium zinc oxide (IZO), tin oxide (SnO₂), or zinc oxide (ZnO), which are transparent and have excellent conductivity.

The compound for the hole injection layer or the hole transport layer is not particularly limited, and may be any compound commonly used for the hole injection layer or the hole transport layer. Non-limiting examples of compounds for the hole injection layer or hole transport layer may include phthalocyanine derivatives, porphyrin derivatives, triarylamine derivatives, and indolocarbazole derivatives. Examples thereof includes 1,4,5,8,9,11-hexaazatriphenylene-hexacarbonitrile (HAT-CN), copper phthalocyanine (CuPc), 4,4′,4″-tris(3-methylphenylamino)triphenylamine (m-MTDATA), 4,4′,4″-tris(3-methylphenylamino)phenoxybenzene (m-MTDAPB), 4,4′,4″-tris(N-carbazolyl)triphenylamine (TCTA), 4,4′,4″-tris(N-(2-naphthyl)-N-phenylamino)triphenylamine (2-TNATA), N4,N4,N4′,N4′-tetra([1,1′-biphenyl]-4-yl)-[1,1′-biphenyl]-4,4′-diamine, bis(N-(1-naphthyl)-N-phenyl)benzidine (α-NPD), N,N′-di(naphthalen-1-yl)-N,N′-biphenyl-benzidine (NPB) or N,N′-biphenyl-N,N′-bis(3-methylphenyl)-1,1′-biphenyl-4,4′-diamine (TPD).

The compound included in the light emitting layer is not particularly limited, and may be any compound commonly used for the light emitting layer. A single light emitting compound or a light emitting host compound may be used.

Here, the light emitting compound in the light emitting layer includes, but is not limited to, a compound capable of emitting light through phosphorescence, fluorescence, thermally activated delayed fluorescence, i.e., TADF (also referred to as E-type delayed fluorescence), triplet-triplet annihilation, or a combination of these processes. The light emitting compound may be selected from a variety of materials depending on the desired emission color. Non-limiting examples of the light emitting compounds include fused ring derivatives such as phenanthrene, anthracene, pyrene, tetracene, pentacene, perylene, naphthopyrene, dibenzopyrene, rubrene, and chrysene, benzoxazole derivatives, benzothiazole derivatives, benzimidazole derivatives, benzotriazole derivatives, oxazole derivatives, oxadiazole derivatives, thiazole derivatives, imidazole derivatives, thiadiazole derivatives, triazole derivatives, pyrazoline derivatives, stilbene derivatives, thiophene derivatives, tetraphenylbutadiene derivatives, cyclopentadiene derivatives, bis(styryl) derivatives, bis(styryl)arylene derivatives, diazaindacene derivatives, furan derivatives, benzofuran derivatives, isobenzofuran derivatives, dibenzofuran derivatives, coumarin derivatives, dicyanomethylenepyran derivatives, dicyanomethylenethiopyran derivatives, polymethine derivatives, cyanine derivatives, oxobenzoanthracene derivatives, xanthene derivatives, rhodamine derivatives, fluorescein derivatives, pyrylium derivatives, carbostyril derivatives, acridine derivatives, oxazine derivatives, phenylene oxide derivatives, quinacridone derivatives, quinazoline derivatives, pyrrolopyridine derivatives, furopyridine derivatives, 1,2,5-thiadiazolopyrene derivatives, pyromethene derivatives, perinone derivatives, pyrrolopyrrole derivatives, squarylium derivatives, violanthrone derivatives, phenazine derivatives, acridone derivatives, deazaflavin derivatives, fluorene derivatives, benzofluorene derivatives, aromatic boron derivatives, aromatic nitrogen boron derivatives, and metal complexes (complexes of metals such as Ir, Pt, Au, Eu, Ru, Re, Ag, and Cu with heteroaromatic ring ligands, etc.). Examples thereof include N1,N1,N6,N6-tetrakis(4-(1-silyl)phenyl)pyrene-1,6-diamine, 2,12-di-tert-butyl-5,9-bis(4-(tert-butyl)phenyl)-7-(3,5-di-tert-butylphenyl)-5,9-dihydro-5,9-diaza-13b-boranaphtho[3,2,1-de]anthracene (t-DABNA-dtB), PtOEP, Ir(ppy)₃, Ir(ppy)₂(acac), Ir(mppy)₃, Ir(PPy)₂(m-bppy), BtpIr(acac), Ir(btp)₂(acac), Ir(2-phq)₃, Hex-Ir(phq)₃, Ir(fbi)₂(acac), fac-tris(2-(3-p-xylyl)phenyl)pyridine iridium (III), Eu(dbm)₃(Phen), Ir(piq)₃, Ir(piq)₂(acac), Ir(Fliq)₂(acac), Ir(Flq)₂(acac), Ru(dtb-bpy)₃·(PF₆)₂, Ir(BT)₂(acac), Ir(DMP)₃, Ir(Mphq)₃, Ir(phq)₂tpy, fac-Ir(ppy)₂Pc, Ir(dp)PQ₂, Ir(Dpm)(Piq)₂, Hex-Ir(piq)₂(acac), Hex-Ir(piq)₃, Ir(dmpq)₃, Ir(dmpq)₂(acac), FPQIrpic, FIrpic, etc.

As the host compound for the light emitting layer, a light-emitting host, a hole-transporting host, an electron-transporting host, or a combination thereof may be used. Non-limiting examples of light-emitting host compounds include fused ring derivatives such as anthracene or pyrene, bis(styryl) derivatives such as bis(styryl)anthracene derivatives or di(styryl)benzene derivatives, tetraphenylbutadiene derivatives, cyclopentadiene derivatives, fluorene derivatives, benzofluorene derivatives, N-phenylcarbazole (9-phenylcarbazole) derivatives, and carbazonitrile derivatives. Non-limiting examples of hole-transporting host materials include carbazole derivatives, dibenzofuran derivatives, dibenzothiophene derivatives, triarylamine derivatives, indolocarbazole derivatives, and benzoxazinophenoxazine derivatives. Non-limiting examples of electron-transporting host materials include pyridine derivatives, triazine derivatives, phosphine oxide derivatives, benzofuropyridine derivatives, and dibenzooxasiline derivatives. Examples thereof include 9,10-bis(2-naphthyl)anthracene (ADN), tris(8-hydroxyquinolinato)aluminum (Alq₃), BAlq (beryllium 8-hydroxyquinolinate), DPVBi (4,4′-bis(2,2-biphenylethenyl)-1,1′-biphenyl) series, spiro-DPVBi (spiro-4,4′-bis(2,2-biphenylethenyl)-1,1′-biphenyl), LiPBO (2-(2-benzoxazolyl)phenol lithium salt), bis(biphenylvinyl)benzene, aluminum-quinoline metal complexes, and metal complexes of imidazole, thiazole, and oxazole.

An electron blocking layer (EBL) may be formed between the hole transport layer and the light emitting layer. The compound for EBL is not particularly limited, and may be any compound commonly used for EBL. For example, the EBL may include N-phenyl-N-(4-(spiro[benzo[d,e]anthracen-7,9′-fluoren]-2′-yl)phenyl)dibenzo[b,d]furan-4-amine), etc.

The compound for the electron injection layer or the electron transport layer is not particularly limited, and may be any compound commonly used for the electron injection layer or the electron transport layer. Non-limiting examples of the compound for the electron injection layer or the electron transport layer include pyridine derivatives, naphthalene derivatives, anthracene derivatives, phenanthroline derivatives, perinone derivatives, coumarin derivatives, naphthalimide derivatives, anthraquinone derivatives, diphenoquinone derivatives, diphenylquinone derivatives, perylene derivatives, oxadiazole derivatives, thiophene derivatives, triazole derivatives, thiadiazole derivatives, metal complexes of oxine derivatives, quinolinol-based metal complexes, quinoxaline derivatives and polymers thereof, benzazole compounds, gallium complexes, pyrazole derivatives, perfluorinated phenylene derivatives, triazine derivatives, pyrazine derivatives, benzoquinoline derivatives, imidazopyridine derivatives, borane derivatives, benzimidazole derivatives, benzoxazole derivatives, benzothiazole derivatives, quinoline derivatives, oligopyridine derivatives such as terpyridine, bipyridine derivatives, terpyridine derivatives, naphthyridine derivatives, aldazine derivatives, carbazole derivatives, indole derivatives, phosphine oxide derivatives, bis(styryl) derivatives, quinolinol-based metal complexes, hydroxazole-based metal complexes, azomethine-based metal complexes, tropolone-based metal complexes, flavonol-based metal complexes, benzoquinoline-based metal complexes, and metal salts. These materials may be used alone or in combination with other materials. Examples thereof include 2-(4-(9,10-di(naphthalen-2-yl)anthracen-2-yl)phenyl)-1-phenyl-1H-benzo[d]imidazole, tris(8-hydroxyquinolinato)aluminum (Alq₃), LiF, Liq, Li₂O, BaO, NaCl, and CsF.

The second electrode (cathode) may include materials such as lithium (Li), aluminum (Al), aluminum-lithium (Al—Li), calcium (Ca), magnesium (Mg), magnesium-indium (Mg—In), and magnesium-silver (Mg—Ag). In addition, for a top-emitting organic light emitting diodes, a transparent cathode may be formed using indium tin oxide (ITO) or indium zinc oxide (IZO) to allow light transmission.

The organic light emitting diode according to an embodiment of the present disclosure may be a top-emitting type or a bottom-emitting type.

The capping layer of the organic light emitting diode according to an embodiment of the present disclosure may have a thickness of about 300 to 1,500 Å, or about 500 to 1,200 Å, or about 600 to 1,000 Å.

The capping layer of the organic light emitting diode according to an embodiment of the present disclosure may have a density of about 1.15 to 1.35 g/cm³, or about 1.2 to 1.3 g/cm³. Within these density ranges, the efficiency of the diode may be further improved.

The organic light emitting diode according to an embodiment of the present disclosure may be used in a display device.

The organic light emitting diode according to an embodiment of the present disclosure may be applied to transparent display devices, mobile display devices, flexible display devices, etc., but is not limited thereto. The capping layer according to the embodiment exhibits high transmittance suitable for transparent display devices and high tensile strength suitable for flexible display devices.

Hereinafter, representative examples of the synthesis methods for the above compounds will be described below. However, the synthesis methods for the compounds of the present disclosure are not limited to the method exemplified below, nor are the implementation of the present disclosure limited to the following Examples and Experimental Examples.

### SYNTHESIS EXAMPLE

Representative Synthesis Example 1 for Compound 1 and Synthesis Example 2 for Compound 2 are exemplarily described, and the compounds represented by Chemical Formula 1 of the present disclosure may be synthesized in the same/similar manner as in Synthesis Example 1 or Synthesis Example 2 below.

In the following reaction schemes, solvents, catalysts, protecting groups, leaving groups, reaction temperatures, reaction times, etc. are representative examples, and any equivalent solvents, catalysts, protecting groups, leaving groups, reaction temperatures, reaction times, etc. may also be used. Synthesis Example 1 below corresponds to a synthesis example that may be used when synthesizing a compound of Chemical Formula 1 of the present disclosure where R₂ is hydrogen, and Synthesis Example 2 corresponds to a synthesis example that may be used when synthesizing a compound of Chemical Formula 1 of the present disclosure where R₂ is not hydrogen. In Synthesis Examples 1 and 2, Reaction Schemes 1 and 2, and Table 6, Reactant 1 and Reactant 2 are notations used to distinguish the two reactants in the synthetic reaction schemes, and Product is a notation used to refer to the final compound synthesized.

### Synthesis Example 1—Synthesis of Compound 1

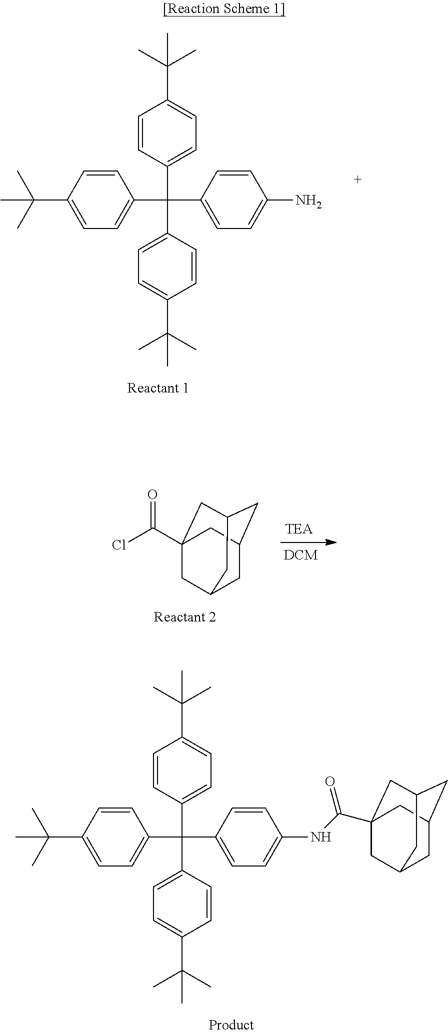

Under a nitrogen atmosphere, Reactant 1 (20 mmol, 10.1 g), Reactant 2 (20 mmol, 4.0 g), and triethylamine (TEA) (100 mmol, 10.1 g) were added to a 500 mL flask, followed by addition of dichloromethane (DCM) (150 mL). The mixture was then stirred at room temperature for 6 hours. After completion of the reaction, the organic layer was extracted with CH₂Cl₂ and water. The extracted solution was treated with MgSO₄ to remove residual moisture, concentrated under reduced pressure, purified by column chromatography, and then recrystallized to afford Compound 1 as Product (90%, 12.0 g).

### Synthesis Example 2—Synthesis of Compound 2

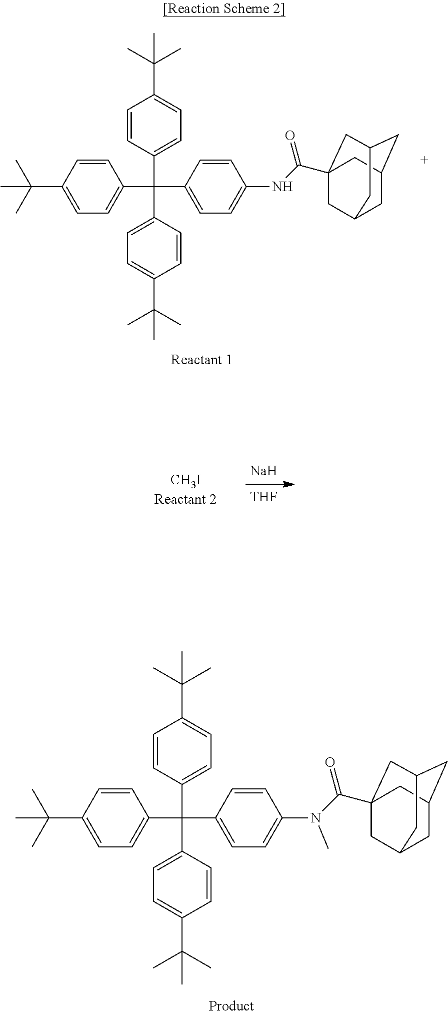

Under a nitrogen atmosphere, Reactant 1 (10 mmol, 6.7 g) and tetrahydrofuran (THF) (100 mL) were added to a 500 mL flask, and sodium hydride (NaH) (40 mmol, 0.96 g) was slowly added while stirring at room temperature for 1 hour. After 1 hour, Reactant 2 (40 mmol, 5.7 g) was slowly added and stirred for 12 hours. After completion of the reaction, the organic layer was extracted using CH₂Cl₂ and water. The extracted solution was treated with MgSO₄ to remove residual moisture, concentrated under reduced pressure, purified by column chromatography, and then recrystallized to afford Compound 2 as Product (95%, 6.5 g).

The representative synthesized compounds (Product), the reactants used (Reactant 1, Reactant 2), the yield, and the [M+H]⁺ results from mass spectrometry (MS) are shown in Table 6 below. The specific compounds of the present disclosure and similar compounds may be synthesized via Synthesis Example 1 or 2.

TABLE 6

| | | | | Amount | |
|---|---|---|---|---|---|
| Item | Reactant 1 | Reactant 2 | Product | obtained (Yield) | [M + H]⁺ |
| P1 | 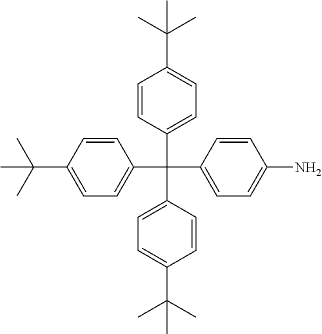 [SMILES: CC(C)(C)c1ccc(C(c2ccc(N)cc2)(c2ccc(C(C)(C)C)cc2)c2ccc(C(C)(C)C)cc2)cc1] | 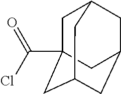 [SMILES: O=C(Cl)C12CC3CC(CC(C3)C1)C2] | 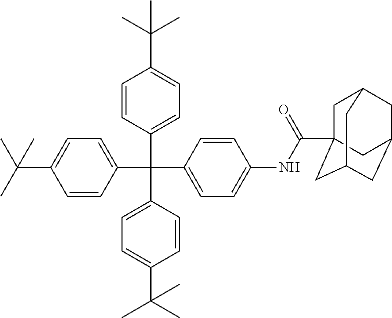 [SMILES: CC(C)(C)c1ccc(C(c2ccc(NC(=O)C34CC5CC(CC(C5)C3)C4)cc2)(c2ccc(C(C)(C)C)cc2)c2ccc(C(C)(C)C)cc2)cc1] Compound 1 | 12.0 g (90%) | 667 |
| P2 | 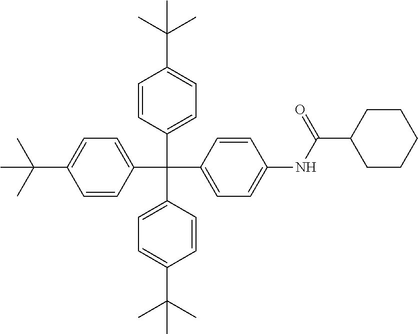 [SMILES: CC(C)(C)c1ccc(C(c2ccc(NC(=O)C3CCCCC3)cc2)(c2ccc(C(C)(C)C)cc2)c2ccc(C(C)(C)C)cc2)cc1] | CH₃I | 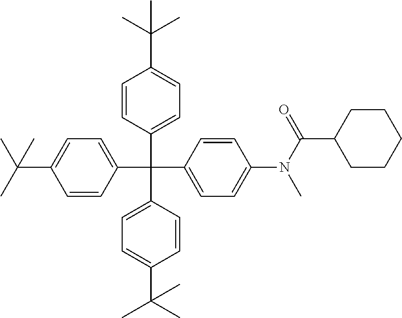 [SMILES: CN(C(=O)C1CCCCC1)c1ccc(C(c2ccc(C(C)(C)C)cc2)(c2ccc(C(C)(C)C)cc2)c2ccc(C(C)(C)C)cc2)cc1] Compound 5 | 5.8 g (93%) | 629 |
| P3 | 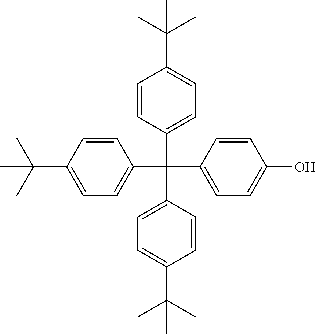 [SMILES: CC(C)(C)c1ccc(C(c2ccc(O)cc2)(c2ccc(C(C)(C)C)cc2)c2ccc(C(C)(C)C)cc2)cc1] | 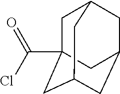 [SMILES: O=C(Cl)C12CC3CC(CC(C3)C1)C2] | 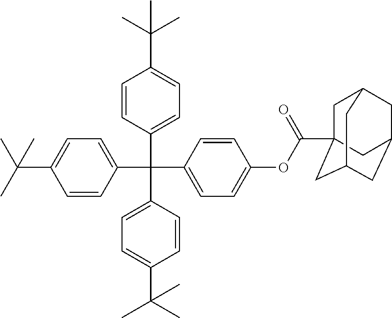 [SMILES: CC(C)(C)c1ccc(C(c2ccc(OC(=O)C34CC5CC(CC(C5)C3)C4)cc2)(c2ccc(C(C)(C)C)cc2)c2ccc(C(C)(C)C)cc2)cc1] Compound 17 | 11.7 g (88%) | 668 |
| P4 | 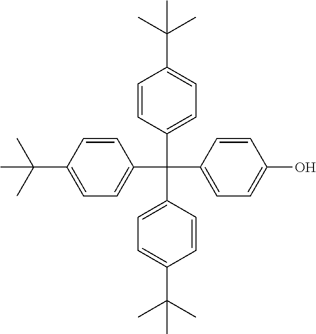 [SMILES: CC(C)(C)c1ccc(C(c2ccc(O)cc2)(c2ccc(C(C)(C)C)cc2)c2ccc(C(C)(C)C)cc2)cc1] | 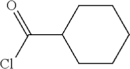 | 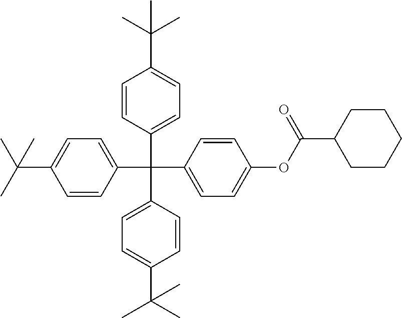 [SMILES: CC(C)(C)c1ccc(C(c2ccc(OC(=O)C3CCCCC3)cc2)(c2ccc(C(C)(C)C)cc2)c2ccc(C(C)(C)C)cc2)cc1] Compound 18 | 11.1 g (90%) | 616 |
| P5 | 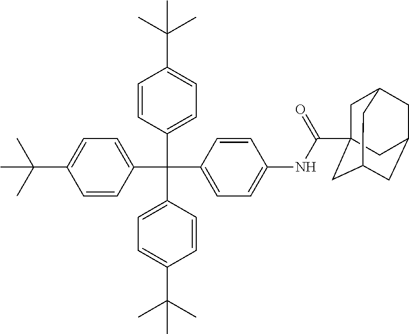 [SMILES: CC(C)(C)c1ccc(C(c2ccc(NC(=O)C34CC5CC(CC(C5)C3)C4)cc2)(c2ccc(C(C)(C)C)cc2)c2ccc(C(C)(C)C)cc2)cc1] | CH₃I | 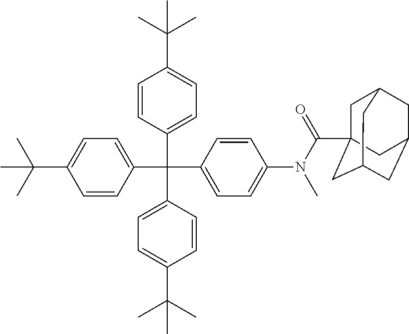 [SMILES: CN(C(=O)C12CC3CC(CC(C3)C1)C2)c1ccc(C(c2ccc(C(C)(C)C)cc2)(c2ccc(C(C)(C)C)cc2)c2ccc(C(C)(C)C)cc2)cc1] Compound 2 | 6.5 g (95%) | 681 |
| P6 | 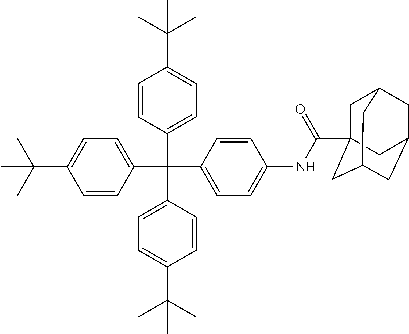 [SMILES: CC(C)(C)c1ccc(C(c2ccc(NC(=O)C34CC5CC(CC(C5)C3)C4)cc2)(c2ccc(C(C)(C)C)cc2)c2ccc(C(C)(C)C)cc2)cc1] | 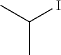 2-iodopropane | 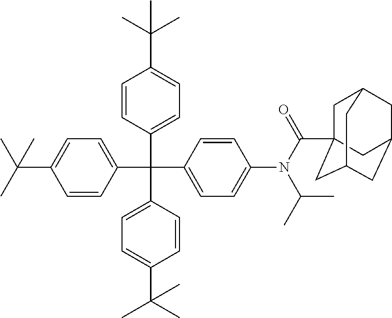 [SMILES: CC(C)N(C(=O)C12CC3CC(CC(C3)C1)C2)c1ccc(C(c2ccc(C(C)(C)C)cc2)(c2ccc(C(C)(C)C)cc2)c2ccc(C(C)(C)C)cc2)cc1] Compound 3 | 6.1 g (86%) | 709 |
| P7 | 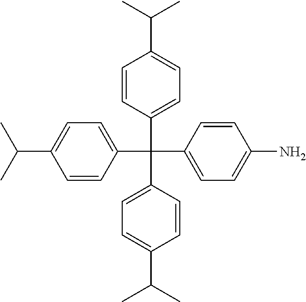 [SMILES: CC(C)c1ccc(C(c2ccc(N)cc2)(c2ccc(C(C)C)cc2)c2ccc(C(C)C)cc2)cc1] | 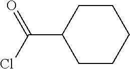 | 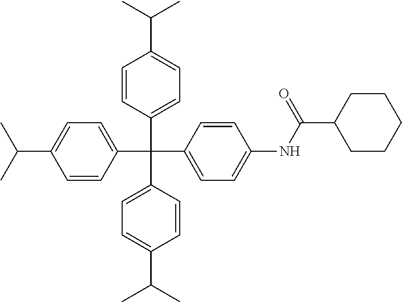 [SMILES: CC(C)c1ccc(C(c2ccc(NC(=O)C3CCCCC3)cc2)(c2ccc(C(C)C)cc2)c2ccc(C(C)C)cc2)cc1] Compound 10 | 10.2 g (89%) | 573 |
| P8 | 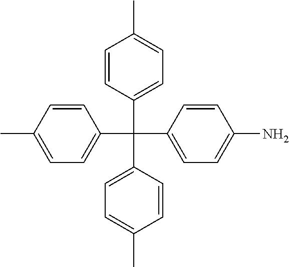 [SMILES: Cc1ccc(C(c2ccc(C)cc2)(c2ccc(C)cc2)c2ccc(N)cc2)cc1] | 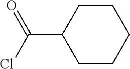 | 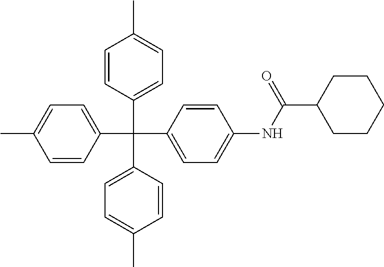 [SMILES: Cc1ccc(C(c2ccc(C)cc2)(c2ccc(C)cc2)c2ccc(NC(=O)C3CCCCC3)cc2)cc1] Compound 9 | 9.0 g (92%) | 489 |
| P9 | 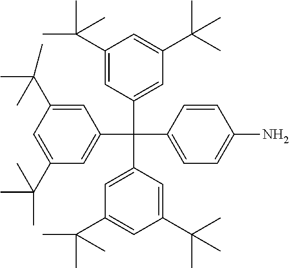 [SMILES: CC(C)(C)c1cc(C(C)(C)C)cc(C(c2ccc(N)cc2)(c2cc(C(C)(C)C)cc(C(C)(C)C)c2)c2cc(C(C)(C)C)cc(C(C)(C)C)c2)c1] | 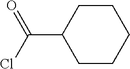 | 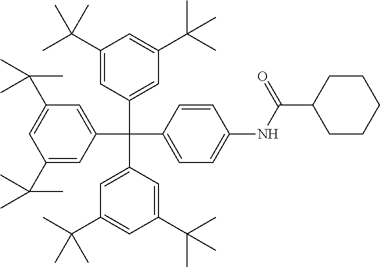 [SMILES: CC(C)(C)c1cc(C(C)(C)C)cc(C(c2ccc(NC(=O)C3CCCCC3)cc2)(c2cc(C(C)(C)C)cc(C(C)(C)C)c2)c2cc(C(C)(C)C)cc(C(C)(C)C)c2)c1] Compound 134 | 13.9 g (89%) | 783 |
| P10 | 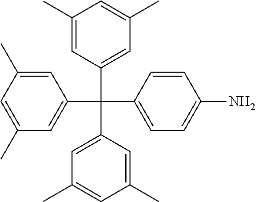 [SMILES: Cc1cc(C)cc(C(c2ccc(N)cc2)(c2cc(C)cc(C)c2)c2cc(C)cc(C)c2)c1] | 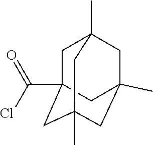 [SMILES: CC12CC3(C)CC(C)(C1)CC(C(=O)Cl)(C2)C3] | 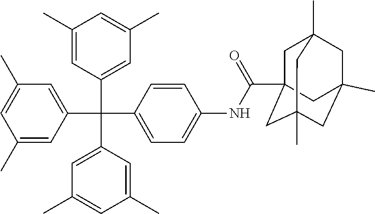 [SMILES: Cc1cc(C)cc(C(c2ccc(NC(=O)C34CC5(C)CC(C)(CC(C)(C5)C3)C4)cc2)(c2cc(C)cc(C)c2)c2cc(C)cc(C)c2)c1] Compound 221 | 11.0 g (88%) | 625 |
| P11 | 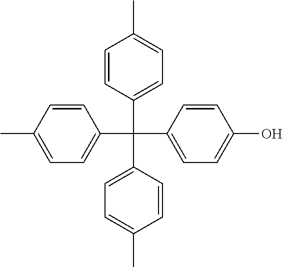 [SMILES: Cc1ccc(C(c2ccc(C)cc2)(c2ccc(C)cc2)c2ccc(O)cc2)cc1] | 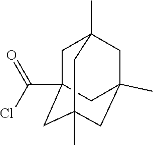 [SMILES: CC12CC3(C)CC(C)(C1)CC(C(=O)Cl)(C2)C3] | 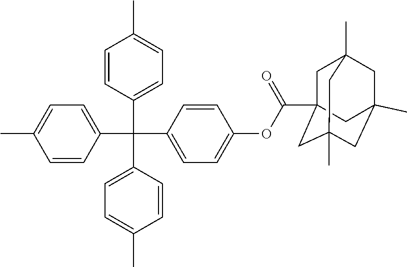 Compound 272 | 10.5 g (90%) | 584 |
| P1 | 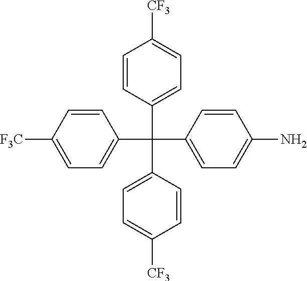 [SMILES: Nc1ccc(C(c2ccc(C(F)(F)F)cc2)(c2ccc(C(F)(F)F)cc2)c2ccc(C(F)(F)F)cc2)cc1] | 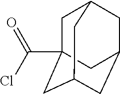 [SMILES: O=C(Cl)C12CC3CC(CC(C3)C1)C2] | 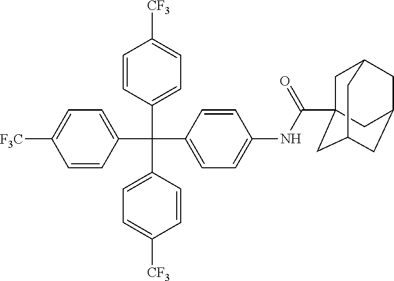 [SMILES: O=C(Nc1ccc(C(c2ccc(C(F)(F)F)cc2)(c2ccc(C(F)(F)F)cc2)c2ccc(C(F)(F)F)cc2)cc1)C12CC3CC(CC(C3)C1)C2] Compound 353 | 12.5 g (89%) | 703 |

### EXPERIMENTAL EXAMPLE

The effects of the compounds of the present disclosure were confirmed through the following experiments. These experiments are merely representative examples and are not limited thereto.

As a representative example, the experiment for confirming the single-film properties (refractive index and transmittance) of Compounds 1 to 4 were described. The compounds represented by Chemical Formula 1 of the present disclosure, which include the same structure as Compounds 1 to 4, may exhibit a similar degree of effect.

### Experimental Example 1—Confirmation of Single-Film Properties (Refractive Index and Transmittance)

To measure optical properties (refractive index and transmittance), a single-film was manufactured by depositing Compound 1 onto a glass substrate (0.7 T) at a deposition rate of 1 Å/sec to a thickness of 1,000 Å under a vacuum of 9×10⁻⁷ Torr.

As shown in Table 7 below, the refractive index and transmittance (%) of the single films manufactured from each of the single-film-forming compounds for optical property evaluation, were measured using an ellipsometer from J.A. WOOLLAM.

TABLE 7

| Single- | | | | | | |
|---|---|---|---|---|---|---|
| film | 460 nm | | 520 nm | | 620 nm | |
| forming | Refractive | Transmittance | Refractive | Transmittance | Refractive | Transmittance |
| material | index | (%) | index | (%) | index | (%) |
| Compound 1 | 1.61 | 100 | 1.60 | 100 | 1.59 | 100 |
| Compound 2 | 1.60 | 100 | 1.59 | 100 | 1.58 | 100 |
| Compound 3 | 1.57 | 100 | 1.57 | 100 | 1.56 | 100 |
| Compound 4 | 1.60 | 100 | 1.59 | 100 | 1.57 | 100 |

Referring to Table 7 above and reviewing the optical properties, it can be confirmed that Compounds 1 to 4 all have a low refractive index of less than 1.70 at wavelengths of 460 nm, 520 nm, and 620 nm. In addition, upon reviewing the transmittance, it can be confirmed that Compounds 1 to 4 all have a high transmittance of 100% at wavelengths of 460 nm, 520 nm, and 620 nm.

### Experimental Example 2—Confirmation of Diode Characteristics

To confirm the diode characteristics of the compounds, Examples and Comparative Examples were prepared as described below.

### Example 1

A substrate on which the ITO (100 nm), serving as the anode of the organic light-emitting diode, was deposited was patterned to be separated into a cathode region, an anode region, and an insulating layer through a photo-lithography process. Then, the anode (ITO) was subjected to UV-ozone and O₂:N₂ plasma surface treatments to increase its work function and to clean the surface.

Next, on the anode, a hole injection layer (HIL) was formed to a thickness of 10 nm using 1,4,5,8,9,11-hexaazatriphenylene-hexacarbonitrile (HAT-CN).

Then, on the hole injection layer, a hole transport layer (HTL) was formed to a thickness of 90 nm by vacuum-depositing N4,N4,N4′,N4′-tetra([1,1′-biphenyl]-4-yl)-[1,1′-biphenyl]-4,4′-diamine. On the hole transport layer, an electron blocking layer (EBL) was formed to a thickness of 15 nm using N-Phenyl-N-(4-(spiro[benzo[d,e]anthracene-7,9′-fluorene]-2′-yl)phenyl)dibenzo[b,d]furan-4-amine.

On the electron blocking layer (EBL), a blue emitting layer was deposited to a thickness of 25 nm using 9,10-bis(2-naphthyl)anthracene (ADN) as the host and 2,12-di-tert-butyl-5,9-bis(4-(tert-butyl)phenyl)-7-(3,5-di-tert-butylphenyl)-5,9-dihydro-5,9-diaza-13b-boranaphtho[3,2,1-de]anthracene (t-DABNA-dtB) as the dopant, wherein the host:dopant weight ratio was 97:3.

On the blue emitting layer, an electron transport layer was formed to a thickness of 25 nm by co-depositing 2-(4-(9,10-di(naphthalene-2-yl)anthracene-2-yl)phenyl)-1-phenyl-1H-benzo[d]imidazole and Liq at a weight ratio of 1:1.

On the electron transport layer, an electron injection layer was formed to a thickness of 1 nm by depositing Liq, and aluminum (Al) was deposited on top of it to a thickness of 100 nm as a cathode.

On the cathode, a high-refractive-index capping layer was deposited to a thickness of 1,000 Å using compound N4,N4′-bis(4-(benzo[d]oxazol-2-yl)phenyl)-N4,N4′-diphenyl-[1,1′-biphenyl]-4,4′-diamine. Then, a low-refractive-index capping layer was deposited to a thickness of 400 Å using Compound 1 of Synthesis Example 1.

Subsequently, a seal cap was bonded to the capping layer (CPL) using a UV-curable adhesive to protect the organic light-emitting diode from oxygen (O₂) or moisture in the atmosphere, thereby manufacturing an organic light-emitting diode according to Example 1.

### Examples 2 to 48

Organic light emitting diodes according to Examples 2 to 48 were manufactured in the same manner as in Example 1, except that the compounds listed in Table 8 were used instead of Compound 1 in the low refractive index capping layer of Example 1.

### Comparative Examples 1 to 3

Organic light emitting diodes according to Comparative Examples 1 to 3 were manufactured in the same manner as in Example 1, except that the following Comparative Compounds 1 to 3 were used instead of Compound 1 in the low refractive index capping layer of Example 1.

### Comparative Compounds 1 to 3

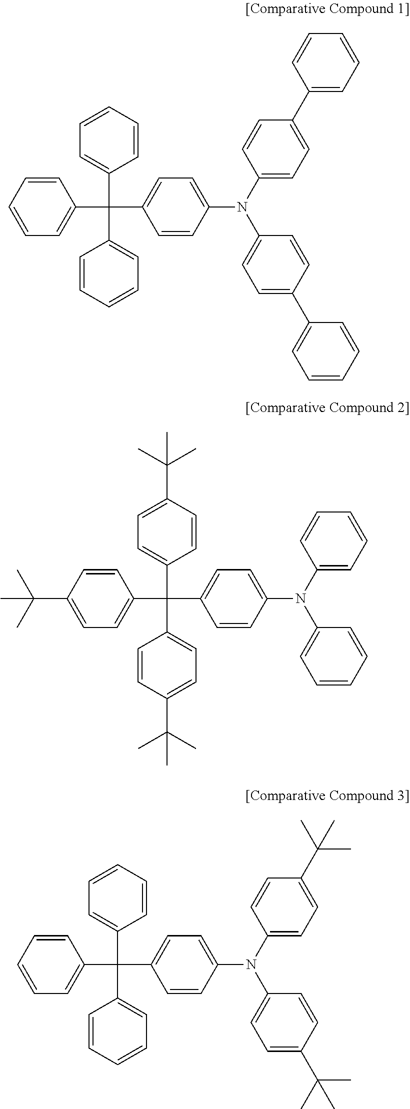 [SMILES: c1ccc(-c2ccc(N(c3ccc(-c4ccccc4)cc3)c3ccc(C(c4ccccc4)(c4ccccc4)c4ccccc4)cc3)cc2)cc1 ; CC(C)(C)c1ccc(C(c2ccc(N(c3ccccc3)c3ccccc3)cc2)(c2ccc(C(C)(C)C)cc2)c2ccc(C(C)(C)C)cc2)cc1 ; CC(C)(C)c1ccc(N(c2ccc(C(C)(C)C)cc2)c2ccc(C(c3ccccc3)(c3ccccc3)c3ccccc3)cc2)cc1]

For the organic light emitting diodes of Examples 1 to 48 and Comparative Examples 1 to 3, the efficiency (Cd/A) was measured by applying a current of 10 mA/cm² using a CS-2000 from KONICA MINOLTA. In addition, the lifetime (LT₉₅) (hrs) was measured as the time required for the luminance to decrease to 95% of its initial value under a constant-current drive of 10 mA/cm² using an M6000 from McScience.

The measurement results are shown in Table 8 below.

TABLE 8

| Examples/ | | | Lifetime |
|---|---|---|---|
| Comparative | Materials for | Efficiency | (LT95) |
| Examples | capping layers | (Cd/A) | (hrs) |
| Example 1 | Compound 1 | 8.29 | 342 |
| Example 2 | Compound 2 | 8.36 | 336 |
| Example 3 | Compound 3 | 8.38 | 338 |
| Example 4 | Compound 4 | 8.27 | 335 |
| Example 5 | Compound 5 | 8.30 | 345 |
| Example 6 | Compound 13 | 8.25 | 337 |
| Example 7 | Compound 26 | 8.30 | 338 |
| Example 8 | Compound 30 | 8.30 | 344 |
| Example 9 | Compound 31 | 8.31 | 340 |
| Example 10 | Compound 32 | 8.31 | 338 |
| Example 11 | Compound 33 | 8.27 | 336 |
| Example 12 | Compound 34 | 8.31 | 343 |
| Example 13 | Compound 8 | 8.23 | 345 |
| Example 14 | Compound 10 | 8.21 | 336 |
| Example 15 | Compound 59 | 8.22 | 342 |
| Example 16 | Compound 65 | 8.25 | 342 |
| Example 17 | Compound 66 | 8.26 | 332 |
| Example 18 | Compound 67 | 8.25 | 333 |
| Example 19 | Compound 7 | 8.19 | 338 |
| Example 20 | Compound 9 | 8.17 | 333 |
| Example 21 | Compound 96 | 8.19 | 342 |
| Example 22 | Compound 102 | 8.20 | 346 |
| Example 23 | Compound 106 | 8.22 | 337 |
| Example 24 | Compound 112 | 8.22 | 343 |
| Example 25 | Compound 133 | 8.43 | 330 |
| Example 26 | Compound 134 | 8.42 | 335 |
| Example 27 | Compound 135 | 8.42 | 345 |
| Example 28 | Compound 173 | 8.43 | 336 |
| Example 29 | Compound 182 | 8.43 | 331 |
| Example 30 | Compound 183 | 8.44 | 336 |
| Example 31 | Compound 192 | 8.44 | 342 |
| Example 32 | Compound 213 | 8.42 | 338 |
| Example 33 | Compound 214 | 8.40 | 339 |
| Example 34 | Compound 221 | 8.42 | 342 |
| Example 35 | Compound 17 | 8.36 | 334 |
| Example 36 | Compound 18 | 8.35 | 338 |
| Example 37 | Compound 19 | 8.33 | 343 |
| Example 38 | Compound 252 | 8.36 | 341 |
| Example 39 | Compound 256 | 8.38 | 341 |
| Example 40 | Compound 257 | 8.38 | 340 |
| Example 41 | Compound 258 | 8.38 | 338 |
| Example 42 | Compound 20 | 8.35 | 339 |
| Example 43 | Compound 259 | 8.32 | 335 |
| Example 44 | Compound 265 | 8.34 | 342 |
| Example 45 | Compound 23 | 8.28 | 341 |
| Example 46 | Compound 270 | 8.29 | 343 |
| Example 47 | Compound 271 | 8.29 | 336 |
| Example 48 | Compound 272 | 8.30 | 335 |
| Comparative | Comparative | 6.16 | 281 |
| Example 1 | Compound 1 | | |
| Comparative | Comparative | 6.89 | 291 |
| Example 2 | Compound 2 | | |
| Comparative | Comparative | 6.88 | 285 |
| Example 3 | Compound 3 | | |

Referring to Table 8, it can be confirmed that the organic light emitting diodes using the compounds according to the Examples exhibit an efficiency greater than about 8.0 Cd/A, whereas the organic light emitting diodes using the compounds according to the Comparative Examples exhibit an efficiency less than about 7.0 Cd/A, demonstrating that the efficiency of the organic light emitting diodes according to the Examples is significantly superior to that of the organic light emitting diodes according to the Comparative Examples.

In addition, regarding lifetime, it can be confirmed that the lifetime of the organic light emitting diodes according to the Examples is relatively longer than that of the organic light emitting diodes according to the Comparative Examples.

### Experimental Example 3—Adhesion Evaluation

To evaluate adhesion, aluminum (Al) serving as the cathode material in Experimental Example 1 was deposited to a thickness of 100 nm on a glass substrate. Thereafter, Compound 1 of Synthetic Example 1 was deposited to a thickness of 400 Å. An adhesive tape was then applied to and removed from the formed low refractive index layer.

As a result, it was confirmed that Compound 1, which has a secondary amide structure in which R₂ is hydrogen, exhibits superior adhesion compared to Compound 2 and Compound 17. In addition, Compound 1 has a structure represented by Chemical Formula 2-1 and thus has n=3, whereas Compound 133 has a structure represented by Chemical Formula 2-2 and thus has n=6. It was confirmed that Compound 1, which is characterized by having a smaller n value and fewer tetraphenyl substitutions, exhibits superior adhesion to an anode material compared to Compound 133.

While the preferred embodiments of the present disclosure have been described in detail above, the scope of the present disclosure is not limited thereto. Various modifications and improvements that may be made by those skilled in the art based on the basic concept of the present disclosure, as defined in the following claims, also fall within the scope of the present disclosure.

What is claimed is:

1. An organic compound represented by the following Chemical Formula 1: 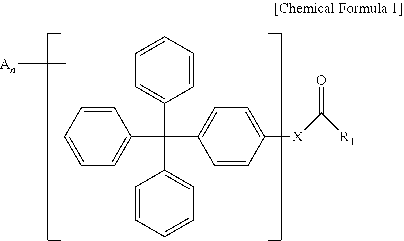
wherein in the chemical formula 1,
n is an integer from 1 to 19,
X is O or N(R₂),
A, R₁, and R₂ are identical to or different from each other, and each of A, R₁, and R₂ is independently selected from the group consisting of hydrogen, a substituted or unsubstituted alkyl group having 1 to 30 carbon atoms, and a substituted or unsubstituted cycloalkyl group having 3 to 20 carbon atoms,
when one or more of A, R₁, and R₂ are substituted, each of A, R₁, and R₂ is independently substituted with one or more substituents selected from the group consisting of deuterium, a cyano group, a halogen group, an alkyl group having 1 to 30 carbon atoms, a cycloalkyl group having 3 to 20 carbon atoms, an aryl group having 6 to 30 carbon atoms, an arylalkyl group having 7 to 30 carbon atoms, a heteroaryl group having 5 to 60 carbon atoms, a heteroarylalkyl group having 6 to 60 carbon atoms, an amine group, an alkylamino group having 1 to 30 carbon atoms, an arylalkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms, and when a plurality of substituents are present, the substituents are identical to or different from each other.

2. The organic compound of claim 1, wherein A, R₁, and R₂ are identical to or different from each other, and each of A, R₁, and R₂ is independently selected from the group consisting of hydrogen, a substituted or unsubstituted alkyl group having 1 to 15 carbon atoms, and a substituted or unsubstituted cycloalkyl group having 3 to 15 carbon atoms.

3. The organic compound of claim 1, wherein n is an integer from 3 to 6.

4. The organic compound of claim 1, wherein A, R₁, and R₂ are identical to or different from each other, and each of A, R₁, and R₂ is independently selected from the group consisting of hydrogen, a substituted or unsubstituted methyl group, a substituted or unsubstituted ethyl group, a substituted or unsubstituted propyl group, a substituted or unsubstituted isopropyl group, a substituted or unsubstituted butyl group, a substituted or unsubstituted sec-butyl group, a substituted or unsubstituted tert-butyl group, a substituted or unsubstituted isobutyl group, a substituted or unsubstituted 2-ethylbutyl group, a substituted or unsubstituted 3,3-dimethylbutyl group, a substituted or unsubstituted pentyl group, a substituted or unsubstituted isopentyl group, a substituted or unsubstituted neopentyl group, a substituted or unsubstituted tert-pentyl group, a substituted or unsubstituted 1-methylpentyl group, a substituted or unsubstituted 3-methylpentyl group, a substituted or unsubstituted 4-methyl-2-pentyl group, a substituted or unsubstituted hexyl group, a substituted or unsubstituted cyclopentyl group, a substituted or unsubstituted tert-butylcyclopentyl group, a substituted or unsubstituted cyclohexyl group, a substituted or unsubstituted tert-butylcyclohexyl group, a substituted or unsubstituted 4-methylcyclohexyl group, a substituted or unsubstituted norbornyl group, and a substituted or unsubstituted adamantly group,
when one or more of A, R₁, and R₂ are substituted, each of A, R₁, and R₂ is substituted with one or more substituents selected from the group consisting of an alkyl group having 1 to 30 carbon atoms, a cycloalkyl group having 3 to 20 carbon atoms, an aryl group having 6 to 30 carbon atoms, an arylalkyl group having 7 to 30 carbon atoms, a heteroaryl group having 5 to 60 carbon atoms, and a heteroarylalkyl group having 6 to 60 carbon atoms, and when a plurality of substituents are present, the substituents are identical to or different from each other.

5. The organic compound of claim 1, wherein A, R₁, and R₂ are identical to or different from each other, and each of A, R₁, and R₂ is independently selected from the group consisting of hydrogen and the structures F1 to F61 below (In the structures below, * indicates a position at which the structure is bonded to the Chemical Formula via a single bond):

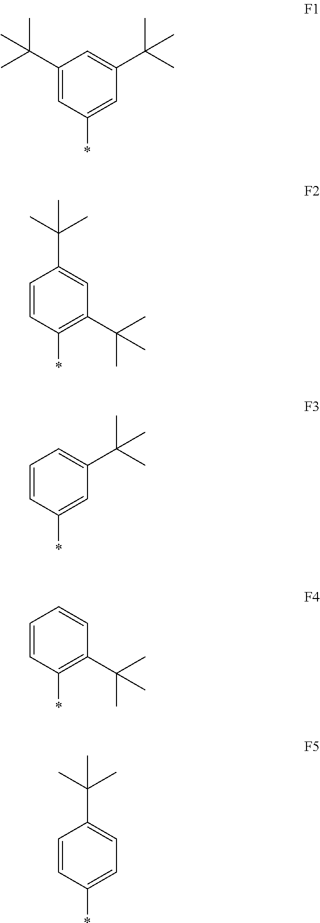 [SMILES: *c1cc(C(C)(C)C)cc(C(C)(C)C)c1 ; *c1ccc(C(C)(C)C)cc1C(C)(C)C ; *c1cccc(C(C)(C)C)c1 ; *c1ccccc1C(C)(C)C ; *c1ccc(C(C)(C)C)cc1]

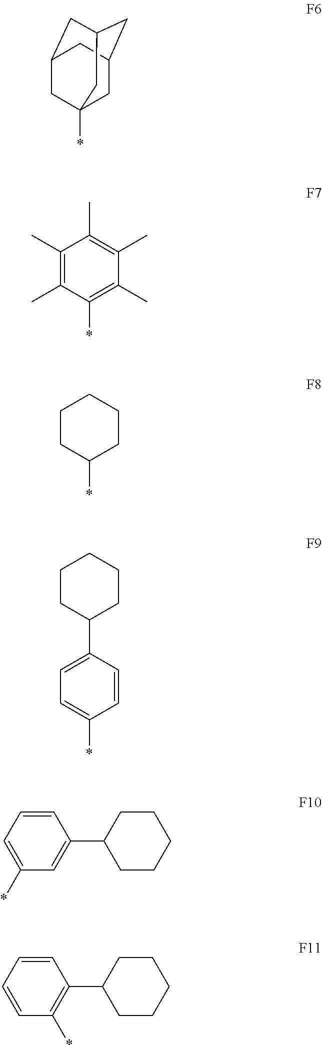

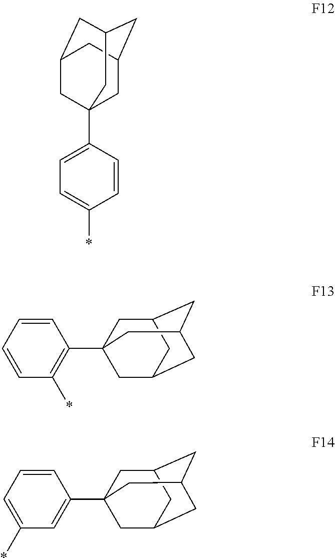 [SMILES: *c1ccc(C23CC4CC(CC(C4)C2)C3)cc1 ; *c1ccccc1C12CC3CC(CC(C3)C1)C2 ; *c1cccc(C23CC4CC(CC(C4)C2)C3)c1]

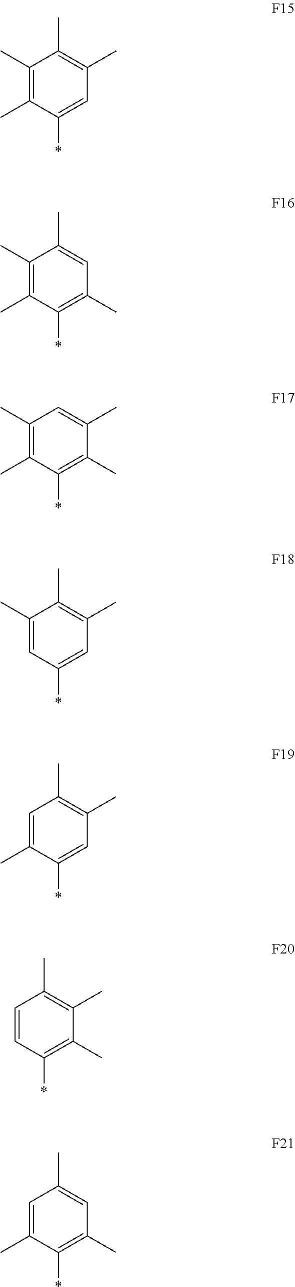

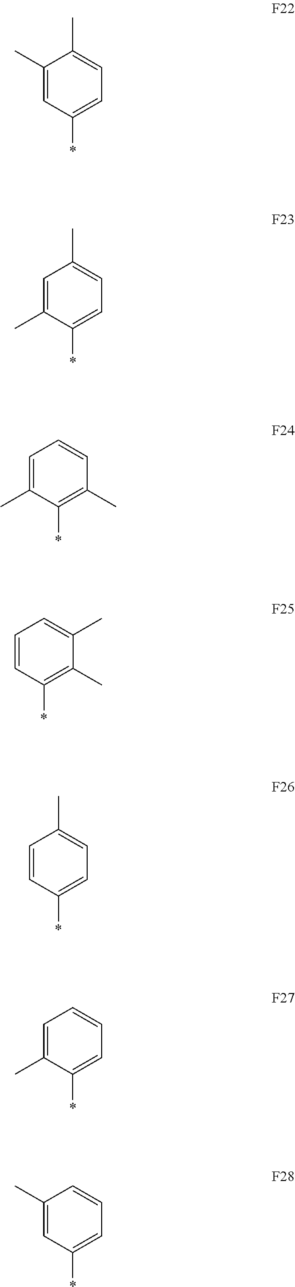

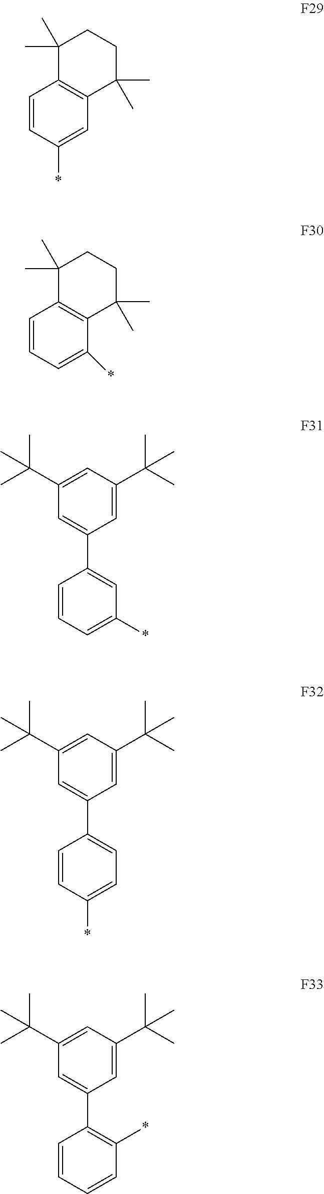

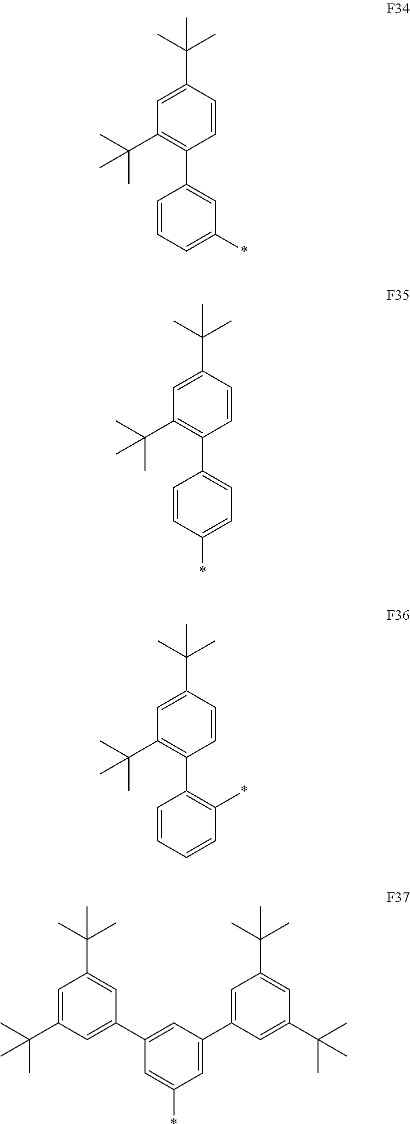 [SMILES: *c1cccc(-c2ccc(C(C)(C)C)cc2C(C)(C)C)c1 ; *c1ccc(-c2ccc(C(C)(C)C)cc2C(C)(C)C)cc1 ; *c1ccccc1-c1ccc(C(C)(C)C)cc1C(C)(C)C ; *c1cc(-c2cc(C(C)(C)C)cc(C(C)(C)C)c2)cc(-c2cc(C(C)(C)C)cc(C(C)(C)C)c2)c1]

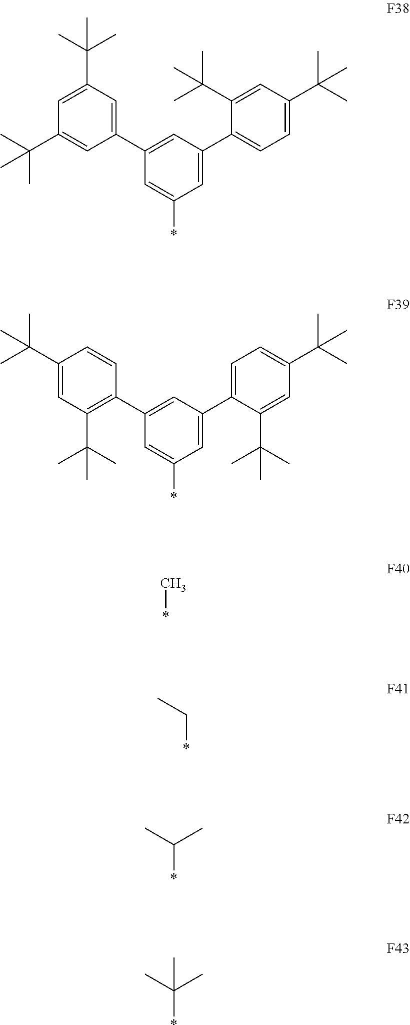

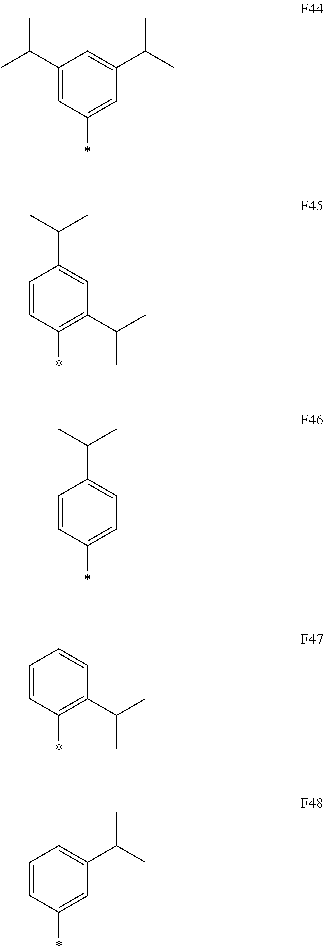

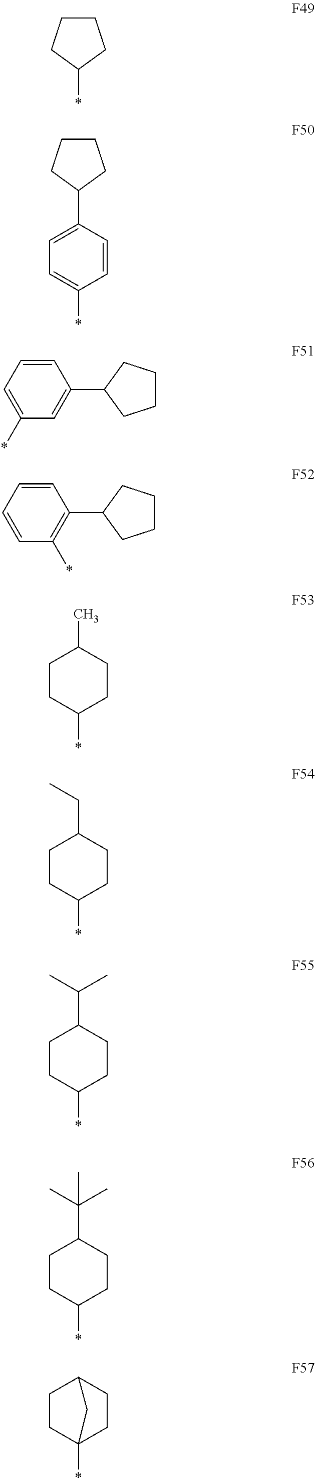

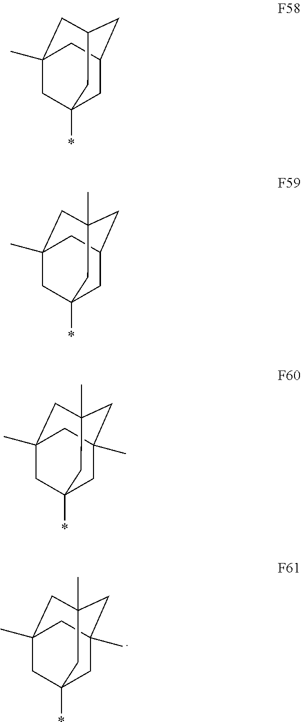

6. An organic light emitting diode comprising:
a first electrode;
a second electrode facing the first electrode;
one or more organic layers disposed between the first electrode and second electrode; and
a capping layer disposed on an outer side of one or more of the first electrode and second electrode,
wherein the capping layer includes the organic compound represented by Chemical Formula 1 according to claim 1.
